(19) United States
(12) Patent Application Publication
(10) Pub. No.: US 2014/0004079 A1
(43) Pub. Date: Jan. 2, 2014

(54) IN VIVO DENDRITIC CELL THERAPEUTIC ADJUVANT

(71) Applicant: Agency for Science, Technology and Research, Singapore (SG)

(72) Inventors: Adam Joseph GEHRING, Singapore (SG); Antonio Bertoletti, Singapore (SG); Florent Ginhoux, Singapore (SG)

(73) Assignee: Agency for Science, Technology and Research, Singapore (SG)

(21) Appl. No.: 13/924,479

(22) Filed: Jun. 21, 2013

Related U.S. Application Data
(60) Provisional application No. 61/662,845, filed on Jun. 21, 2012.

(51) Int. Cl. A61K 39/00

(52) U.S. Cl. CPC A61K 39/0005
USPC 424/85.2; 424/184.1; 424/130.1; 424/85.1; 424/85.5; 424/85.7; 424/85.6; 530/351; 530/387.1; 530/350; 536/23.1; 536/1.11; 554/1

(57) ABSTRACT

Disclosed is a method of eliciting an immune response to an antigen present endogenously in a mammal. The method may comprise administering to the mammal a composition comprising at least one immunomodulator for inducing cell differentiation and or antigen-presenting function of antigen-presenting cell precursor. The antigen-presenting cell precursor may have taken up the antigen. Also disclosed are a composition for use in the method, an adjuvant comprising the composition, the use of the composition and the immunomodulator as described herein.

### CROSS-REFERENCE TO RELATED APPLICATIONS

This application claims the benefit of priority of U.S. provisional application No. 61/662,845, filed 21 Jun. 2012, the contents of it being hereby incorporated by reference in its entirety for all purposes.

### FIELD OF THE INVENTION

The present invention generally relates to the field of immunology. In particular, the present invention relates to an adjuvant.

### BACKGROUND OF THE INVENTION

Therapeutic vaccination for chronic infections, be it recombinant antigens, peptides, viral vectors, DNA or dendritic cells are hindered by the need to select appropriate antigens. It is a major complicating factor due to the evolutionary diversity that pathogens have developed in response to selective forces exerted by individual (immune response) or environmental (drugs, vectors) factors. Moreover, peptides covering conserved regions for vaccination are HLA restricted and can only be applied to selected patients with the appropriate HLA. As a result, recombinant antigens or DNA vectors coding pathogen proteins may misdirect the intended immune response due to differences between the infectious pathogen and the antigen sequence utilized for vaccination.

A hallmark of many chronic infections is the constant production of pathogen proteins. This is particularly evident in hepatitis B virus (HBV) infection, where viral titers can reach 10⁹-10¹⁰ virions/ml in the serum. The HBV surface antigen (HBsAg) is produced in excess of whole virions and reaches concentrations well into the μg/ml range. While persistently present viral antigen is generally considered a negative factor, the abundance of endogenously produced viral antigen could be internalized by different cell types Proper activation of cells internalizing antigen in the circulation of chronic patients could provide a target for therapeutic vaccination and stimulate T cells with antigen customized to the patient's viral genome.

Accordingly, there is a need to provide an improved method and composition for eliciting, an immune response.

### SUMMARY OF THE INVENTION

In one aspect, there is provided a method of eliciting an immune response to an antigen present endogenously in a mammal. The method may comprise administering to the mammal a composition comprising at least one immunomodulator in an amount sufficient for inducing cell differentiation and/or antigen-presenting function of an antigen-presenting cell precursor. The antigen-presenting cell precursor may have taken up the antigen.

In another aspect, there is provided a composition for eliciting an immune response to an antigen present endogeneously in a mammal. The composition may comprise at least one immunomodulator in an amount sufficient in inducing cell differentiation and/or antigen-presenting function of an antigen-presenting cell precursor. The antigen-presenting cell precursor may have taken up the antigen.

In another aspect, there is provided an adjuvant that may comprise the composition as described herein.

In another aspect, there is provided the use of at least one immunomodulator in the manufacture of a medicament for eliciting an immune response to an antigen present endogenously in a mammal. The at least one immunomodulator may be in an amount sufficient for inducing cell differentiation and/or antigen-presenting function of an antigen-presenting cell precursor. The antigen presenting cell precursor has taken up the antigen.

In another aspect, there is provided an immunomodulator that is capable of eliciting an immune response to an antigen present endogenously in a mammal. The immunomodulator may be in an amount sufficient for inducing cell differentiation and/or antigen-presenting function of an antigen-presenting cell precursor. The antigen-presenting cell precursor may have taken up the antigen.

### BRIEF DESCRIPTION OF THE DRAWINGS

The invention will be better understood with reference to the detailed description when considered in conjunction with the non-limiting examples and the accompanying drawings, in which:

FIG. 1 shows flow cytometry antigen presenting cells (APC) gating strategy and population frequency analysis in healthy donors and chronic HBV patients. A) Lineage negative HLA-DR+ APCs were derived from total live PBMC gated by forward and side scatter followed by single cell gating using width & height parameters with viability dye (data not shown). CD11c myeloid DC, CD141 DC, CD123 pDC and CD20 B cells were identified from the CD14/CD16 double negative population. B) Frequency of HLA-DR+ cells in the total PBMC and the frequency of specific APC populations as a proportion of the HLA-DR+ cells. There were no statistically significant difference determined by 1 way ANOVA and Tukey post-test analysis using total CHB patients and patient categories (FIGS. 10 and 11). FIG. 1 compares the profiles of the various APCs lineages in healthy donors and chronic HBV patients.

FIG. 2 is a series of scatter plots of division index (A) and proliferation index (B) of T cell proliferation following co-culture with chronic HBV patient APCs. A) Frequency of T cells induced to proliferate with each APC population in healthy subjects or different HBV cohorts (HBV Hi>10⁶ copies/ml; HBV low ≦10⁶ copies/ml; Flare ALT 500 U/L). B) Normalized proliferation index with each APC population. There were no statistically significant differences. FIG. 2 shows APCs in chronic HBV patients and healthy patients have the same capabilities in inducing T cells to proliferate or divide.

FIG. 3 is the results of the study on the characterisation of HBsAg staining in FACs sorted APC populations. A) Dot plot showing quantitative data derived from fluorescent images acquired on Tissue Faxs system. Tissue Guest 3.0 was used to analyze each cell for the presence of HBsAg and plotted on dot plot. Dot plots for one representative chronic HBV patient (CHB; top row) and one healthy donor (bottom row) are presented. B) Compilation of the frequency of HBsAg+ cells from multiple patients for each APC population. C) Examination of the correlation of HBsAg positive staining with viral load in CD14 MN cells. D) Study shows that reducing viral load with antiviral therapy does not alter HBsAg staining in CD14 MN E) Analysis HBsAg staining on CD14 MN cells from five chronic HBV patients with increasing HBsAg in their serum. FIG. 3 shows APC precursor cell CD14MN contain HBsAg antigen and that a dose dependent increase of HBsAg+ CD14 occurs as HBsAg level in serum increases.

FIG. 4 is a series of bar graphs showing showing that there is no ex vivo cross-presentation by the various APCs. Representative s183-TCR T cell IFN-γ Elispot following overnight co-culture with A) mDC, CD14 MN, CD16 MN & B cell; B) pDC; C) CD141 DC sorted from a representative chronic HBV patient. APCs were left untreated (Ø) or activated overnight with TLR ligands, IFN-α or IFN-γ then co-cultured with s183-TCR T cells. All cell types were able to present exogenously added peptide. Data in panel A is representative of seven patients and panels B & C are representative of three patients. FIG. 4 shows that whilst no constitutive or inducible ex vivo cross-presentation of HBsAg was detected in the six different APC populations tested, CD141 DC can efficiently cross-presented recombinant HBsAg loaded exogenously when stimulated with polyI:C.

FIG. 5 shows bar graph results of studies into the possibility of CD 14 MNs differentiating to DC, which then would facilitate cross-presentation of captured HBV antigen. CD14 MN from a healthy HLA-A0201+ donor were either loaded with HBV antigen for 4 h at day zero or left untreated. Following antigen loading, they were differentiated with different combinations of cytokines to determine if the differentiation program altered their ability to cross-present antigen. CD14 MNs were cultured with 750 U/ml GM-CSF alone for 3 and 6 d; 750 U/ml GM-CSF+1500 U/ml IL-4 for 6 d (GM/4); 750 U/ml GM-CSF+200 ng/ml IL-15 for 6 d (GM/15; Mohamadzadeh et. Al., J. Exp. Med., 2001); 750 U/ml GM-CSF+1000 U/ml IFN-α for 3 d (GM/α, Santini et al, J. Exp. Med, 2000) ; or 750 U/ml GM-CSF+500 U/ml IFN-γ for 6 d (GM/γ; Delneste et. al., Blood, 2003); or 100 ng/ml IL-32 for 6 d and cross-presentation of antigen captured at day zero was tested with HBV-specific TCR-redirected T cells in IFN-γ elispot A) without any additional activation or B) after activation with LPS & CD40L. All of the differentiation programs resulted in cross-presentation of antigen captured by CD14 MN to different degrees, including GM-CSF alone for 3 d. Activation of moDC increased the T cell response in all conditions. Thus, multiple differentiation programs can mobilize monocytes to cross-present in vivo captured antigen. FIG. 5 shows CD14 monocytes, which is an antigen-presenting cell precursor cell, when differentiated according to the method of the present disclosure could cross-present HBV antigen.

FIG. 6 shows the characterisation of monocyte-derived DC from patients' ability to cross-present in vivo captured HBsAg. A) Frequency of HBsAg+ CD14 MN prior to differentiation to monocyte-derived dendritic cells. B) Cross-presentation of circulating, in vivo captured HBsAg following co-culture of s183-TCR and s171-TCR CD8 T cells with differentiated moDC in the absence of activation. Healthy donor and HLA-mismatched chronic HBV patient moDC served as negative controls. Positive responses in HLA matched moDC are colored in black. C) Activation of moDC can increase the T cell response. Monocyte-derived DC were activated overnight with polyI:C or LPS+CD40L and then co-cultured with s183-TCR and s171-TCR CD8 T cells. Thus, FIG. 6 shows monocyte-derived DCs from patients, when differentiated, are capable of cross-presenting in vivo captured HBsAg.

FIG. 7 shows results of the studies on T cell expansion of autologous T cells by moDC presenting in vivo captured antigen. A) IFN-γ elispot for chronic HBV patient virus-specific T cells expanded with pools of synthetic peptides covering HBcAg (1 pool=core, 42 peptides) and HBsAg (2 pools=Env1 & Env2; 42 peptides each). Data displayed as fold above background to normalize variation between patients due to varying background for each assay. Positive cutoff was >10 spots and 2 times the background. B) IFN-γ elispot for chronic patient virus-specific T cells after expansion with moDC made using GM-CSF+IL4. C) IFN-γ elispot for chronic patient virus-specific T cells after expansion with moDC made using GM-CSF+IL-15. Positive responses for moDC were defined as greater than two times the average of unstimulated wells and ≧10 spots. Background and responses for the moDC assays were lower than peptide expanded cultures and thus are presented as IFN-γ spots/10⁵ cells. Intracellular cytokine staining to confirm D) CD8 and E) CD4 HBV-specific T cell expansion with moDC presenting in vivo captured antigen. Cytokine staining are responses from 2 separate patients and representative of 4 patients where responses could be detected by intracellular staining, *=positive response; nt=not tested. Thus, FIG. 7 demonstrates that the administration of an immunomodulator to antigen-presenting precursor cells that have taken up antigen (i.e. HBsAg+ CD14 MN) can elicit immune response.

FIG. 8 is a series of studies on circulating CD141 DC and pDC in chronic HBV patients. A) CD141+ DC and CD123+ pDC were identified from the lineage negative, HLA-DR+, CD14/CD16 negative population and positive staining for CD141 and CD123. B) Frequency of CD141 DC and pDC as a proportion of the HLA-DR+ cells in healthy donors and chronic HBV patients. C) Frequency of HBsAg+ pDC (top row) and CD141 DC (bottom row) from at least 4 different donors. D) IFN-γ elispot using ex vivo sorted pDC stimulated overnight with TLR ligands and IFN-α and co-cultured with CD8 enriched s183-TCR T cells to determine if they can present in vivo captured HBsAg. CD123+ pDC were also loaded with rHBsAg and activated with immunomodulator TLR ligands to determine if they have the capacity to cross-present soluble HBsAg. Peptide loaded pDC demonstrate they can activate s183-TCR T cells. E) IFN-γ elispot using ex vivo sorted CD141 DC stimulated overnight with immunomodulator polyI:C and co-cultured with CD8 enriched s183-TCR T cells to determine if they can present in vivo captured HBsAg. CD141 DC were also loaded with rHBsAg and activated to with polyI:C to demonstrate they have the capacity to cross-present soluble HBsAg. Data shown is representative of 3 different patients. Thus, FIG. 8 shows that constitutive or inducible ex vivo cross-presentation of HBsAg does not occur in the specialized CD141 DC or pDC of chronic HBV patients but the ability of CD141 DC to cross-present soluble antigen is not altered by HBV persistence.

FIG. 9 shows the results of study on cross-presentation by healthy donor moDC loaded prior to differentiation. A) Isolated CD14 MN were loaded with increasing concentrations of rHBsAg for 4 h prior to differentiation and stained for HBsAg to confirm uptake. B) Differentiated moDC that were loaded at 0 d were co-cultured with s183-TCR CD8 T cells +/−LPS/CD40L activation and cross-presentation was monitored by T cell activation in IFN-γ elispot. FIG. 9 shows activation by the method or composition of the present disclosure enhances or elicits T cells response.

FIG. 10 shows the results on the frequencies of APC populations. The frequency of total HLA-DR+ cells were calculated for each healthy donor and chronic HBV patient. The different APC populations were then calculated as a percentage of the total HLA-DR+ cells to normalize between patients. Chronic HBV patients were segregated based on the level of alanine amino transferase (ALT) in their serum, an enzyme released upon liver damage and inflammation. Patients were grouped into ALT normal and ALT high; defined as >120 U/L or greater than 2 times upper limit of normal (40 U/L). Thus, FIG. 10 demonstrates that the frequencies of APC populations in chronic HBV patients are not significantly affected by liver inflammation.

FIG. 11 shows that the frequency of APC populations is not significantly affected by viral load. Frequency of HLA-DR+ cells in the total PBMC and the frequency of specific APC populations as a proportion of the HLA-DR+ cells. Patients were categorized based on the level of HBV DNA; HBV low ≦10⁶ copies ml and HBV hi >10⁶ copies/ml. There were no statistically significant difference determined by 1 way ANOVA and Tukey post-test analysis.

FIG. 12 shows the study on the characterisation of the HBV-specific T cell receptor (TCR) redirected T cells used for functional cross-presentation assays. A) Primary human T cells from a single healthy donor were transduced with either HLA-A2 restricted Hbs183-91-(s183-TCR) or Hbc18-27-specific (c18-TCR) or HLA-Cw08 restricted Hbs171-80-specific (s171-TCR) TCRs. TCR expression was monitored using matching HLA-A2 pentamers (s183-TCR & c18-TCR) and HLA-C08 tetramers kindly provided by Gijsbert Grotenbreg at the National University of Singapore. Clear pentamer/tetramer positive populations were evident, compared to mock transduced T cells, for all three TCRs. B) Functional profile of s183-TCR T cells. S183-TCR T cells were stimulated overnight with HLA-A2+T2 loaded with 1 μg/ml HBs183-91 peptide in the presence of 2 μg/ml Brefeldin A. Cells were then stained with antibodies for CD3, CD4, CD8, IFN-γ, IL-2, TNF-α, IL-22, IL-17, IL-21, IL-8, Mip1⊖, IL-4 and IL-10. Unpulsed T2 cells (top row) show the background production of each cytokine. Peptide specific activation (bottom row) demonstrates that these transduced T cell populations can make at least 7 different cytokines. IFN-γ was chosen to monitor cross-presentation in functional assays because it was the dominant cytokine produced by TCR redirected CD8 T cells. FIG. 12 demonstrates that T cells activated in accordance with the method or composition of the present disclosure have normal T cell functions and secretion.

FIG. 13 is a bar graph that shows confirmation of specificity of the T cell response using different TCR redirected T cells. CD14 MN from an HLA=A0201 healthy donor were loaded at day zero with the HBV antigens indicated in the graph and then differentiated with GM-CSF and IL-4 for 6 days to generate moDC. Following differentiation, moDC were co-cultured with HBcAg-specific TCR redirected T cells (c18-TCR) overnight for IFN-γ Elispot without the addition of any exogenous antigen. Results demonstrate that there was no non-specific T cell activation due to HBsAg loading in 0 d CD14 MN and that cross-presentation due to CD14 MN differentiation also occurred for the HBcAg, which indicates it can be expanded to other HBV antigen. Thus, FIG. 13 demonstrates that the method of the present disclosure may be used to elicit immune response to other HBV antigens.

### BRIEF DESCRIPTION OF THE TABLES

Table 1 shows patient characteristics for population analysis.

Table 2 shows patients characteristics for MLR experiments.

Table 3 shows patients characteristics for CD14 MN HBsAg staining.

Table 4 shows patient characteristics for ex vivo cross-presentation.

Table 5 shows patient characteristics for moDC cross-presentation experiments.

Table 6 shows patient characteristics for autologous expansions.

### DETAILED DESCRIPTION OF THE PRESENT INVENTION

Selection of the correct antigens for therapeutic vaccination against chronic infections is complicated by pathogen and host genetic variations. In many chronic infections, pathogenic antigens are constantly produced in abundance. The persistently present viral antigen is generally considered a negative factor. However, the inventors of the present disclosure found a way of utilising this abundance of antigens in circulation as a way of providing therapeutic vaccination. In the present disclosure, endogenous antigen present during persistent infections was found to advantageously provide a personalised antigen repertoire for therapeutic vaccination when harnessed for efficient presentation to T cells.

Thus, in one aspect, there is provided a method of eliciting an immune response to an antigen. In one example, the method may elicit an immune response to an antigen that is present endogenously in a mammal. The method as described herein comprises administering to the mammal a composition comprising at least one immunomodulator in an amount sufficient for inducing cell differentiation and/or antigen-presenting function of an antigen-presenting cell precursor. The antigen-presenting cell precursor has taken up or incorporated the antigen.

Also disclosed is a composition for eliciting an immune response to an antigen present endogenously in a mammal. In one example, the composition comprises at least one immunomodulator in an amount sufficient for inducing cell differentiation and/or antigen-presenting function of an antigen-presenting cell precursor. In one example, the antigen-presenting cell precursor has taken up or incorporated the antigen.

In one example, the method as disclosed herein requires no exogenous antigen to be administered to the mammal with the composition. That is, the composition as disclosed herein does not contain exogenous antigen.

As used herein, the term “taken up” refers to the process of internalization of antigen by antigen-presenting cells. The internalization of antigen by antigen-presenting cells or antigen-presenting cells precursor may occur either by phagocytosis or receptor-mediated endocytosis. Upon internalization, the antigen would then be processed by the antigen-presenting cells or antigen-presenting cells precursor for display or expression on MHC class I or II molecules. Lymphocytes that recognize the antigen presented on the MHC class I or II would then be activated, thus the immune response is elicited. Accordingly, in one example, the process of internalisation of antigen by antigen-presenting cell or antigen-presenting cell precursor of the present disclosure occurs in vivo prior to the isolation of the antigen-presenting cell or antigen-presenting cell precursor or prior to the administration of the composition of the present disclosure. In one example, the process of internalisation of antigen by antigen-presenting cell or antigen-presenting cell precursor does not occur ex vivo.

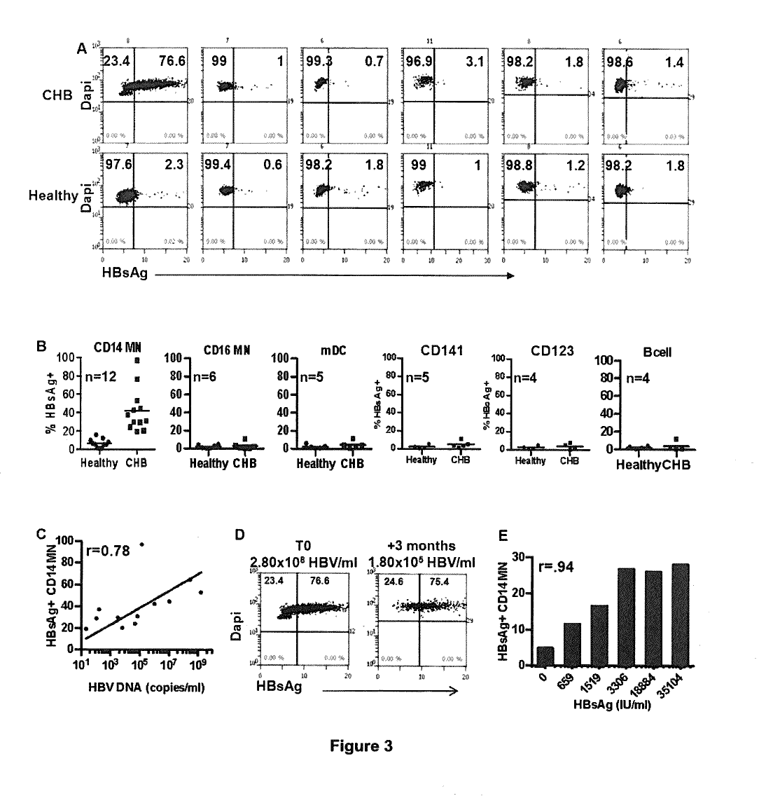

One example of antigen-presenting cell precursor such as CD14 MNs presenting endogenous antigens found in chronic Hepatitis B patients can be found in the Experimental Section, for example, FIG. 3. Thus, the method and/or composition of the present disclosure elicits and/or enhances disease-specific T cell responses without being hindered by the need to select appropriate antigens and a GMP cell production facility to produce the vaccine. In other words, the method of the present disclosure enables the administration of only adjuvants for therapeutic vaccination, whilst relying on endogenous pathogenic antigen present in chronically infected patients.

As used herein, the term “endogenous” when used in reference to antigen in the present disclosure refers to antigen that is present in the body or in vivo. In one example, when the antigen is taken up or internalised in vivo, the antigen is considered endogenous antigen. In one example, the endogenous antigen may be found within the body with chronic infection. Accordingly, the term “exogenous” when used in reference to antigen in the present disclosure refers to antigen that is provided outside of the subject. That is, exogenous antigen refers to antigens that are introduced to antigen-presenting cell precursor in vitro. In one example, when the antigen is taken up or internalised ex vivo or in vitro, the antigen is considered exogenous antigen.

As used herein, the term “elicit” refers to the act of drawing forth, initiating or evoking an immune response in the mammal. The term “elicit” in the present disclosure may be used as a synonym of “enhancing”.

As used herein, the term “immune response” refers to the system of biological structures and processes within an organism that protects against disease. In one example, the immune response in the present disclosure refers to the activation of lymphocytes in response to presentation of antigen to the cells by antigen-presenting cells. In one example, the lymphocytes may be T cells or B cells. In one example, the lymphocytes may be T cells and may include, but are not limited to, CD4, CD8 or NK T cells. In one example, the lymphocytes may be CD8 T cells.

As used herein, the term “immunomodulator” refers to agents that exert modifying or controlling influence on the immune system. An “immunomodulator” may induce, enhance or suppress the immune response. In the present disclosure, the immunomodulator preferably induces or enhances the immune response. In one example, the immunomodulator preferably induces cell differentiation and/or antigen-presenting function of an antigen-presenting cell precursor. In one example, the immunomodulator may include, but is not limited to, a colony stimulating factor, a cytokine, a nucleotide, a tumour necrosis factor, a transforming growth factor, an antibody, such as anti-LILRA2, a recombinant receptor ligand, a chemokine, a carbohydrate, a lipid, a pathogen associated molecular pattern (PAMP); an endogenous danger-associated molecular pattern (DAMP), a CD40 ligand, an ALUM (AB(SO₄)₂.12H₂O) (A is an element selected from Na and K; B is an element selected from Al and Cr), or a combination thereof.

The colony stimulating factor as used in the present disclosure is a cytokine that functions as a white blood cell growth factor and may include, but is not limited to, granulocyte-macrophage colony-stimulating factor (GM-CSF), granulocyte colony-stimulating factor (G-CSF), a macrophage colony-stimulating factor (M-CSF or CSF-1), or a combination thereof. In one example, the cytokine as used in the present disclosure may include, but is not limited to, interleukin-1 (IL-1), IL-2, IL-3, IL-4, IL-6, IL-10, IL-11, IL-12, IL-15, IL-18, IL-32, interferon-α (IFN-α), IFN-β, IFN-γ, or a combination thereof.

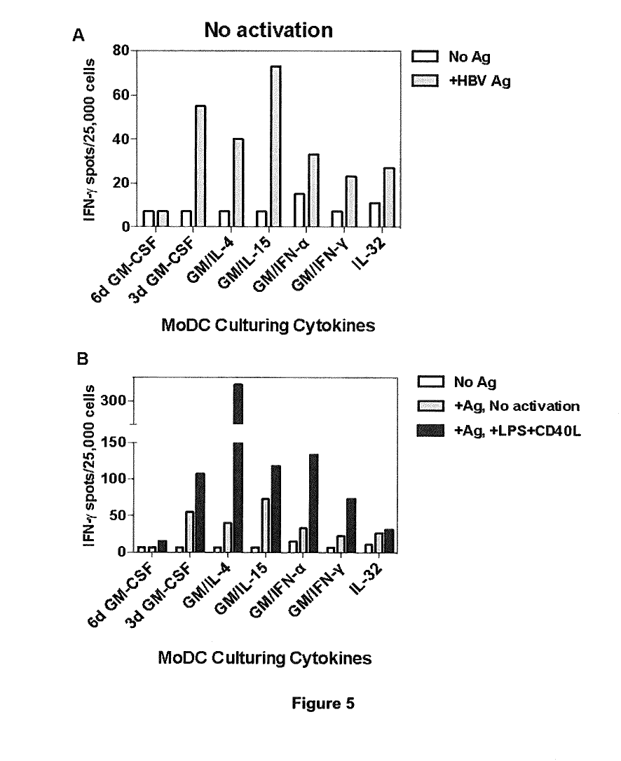

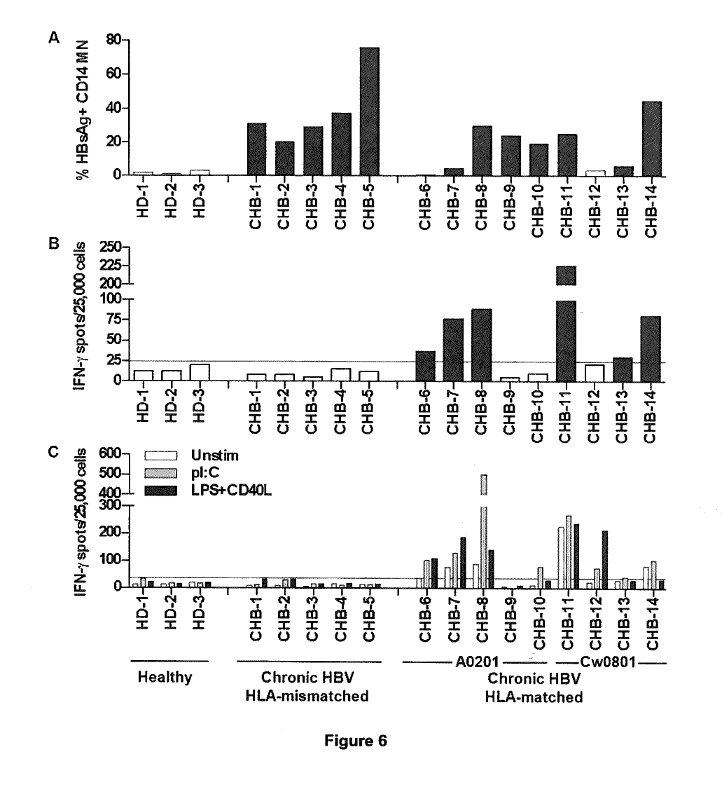

In one example, the composition of the present disclosure may comprises at least two, three, four, five, six, seven, eight, nine, ten, eleven, twelve, thirteen, fourteen, fifteen, sixteen, seventeen, eighteen or all of the immunomodulators. In one example, the composition of the present disclosure may comprise at least two of the immunomodulators, including, but not limited to, the following combinations: (a) GM-CSF and IL-4; (b) GM-CSF and IFN-α; (c) GM-CSF and CpG; (d) GM-CSF and IL-15; (e) GM-CSF and IFN-γ; (t) GM-CSF and IL-32; (g) GM-CSF and LILRA2; (h) GM-CSF and Alum; or (i) GM-CSF and CD40L. In one example, the composition of the present disclosure may comprise at least two of the immunomodulators, including, but not limited to GM-CSF and IL-4; GM-CSF and IL-15; GM-CSF and IFN-α; GM-CSF and IFN-γ; or GM-CSF and IL-32, which as shown in the Experimental Section, for example FIGS. 5 and 6, such combinations elicit immune response.

In one example, a pathogen associated molecular pattern (DAMP) may include, but is not limited to, such as a synthetic Toll-like receptor ligand, a synthetic Toll-like receptor agonist, a natural Toll-like receptor ligand or a natural Toll-like receptor agonist.

In one example, the nucleotide as used in the present disclosure may include, but is not limited to, a nucleotide comprising a CpG motif that is recognized by TLR-9, such as ODN 2006 (5′-tcgtcgtttistcgttttgtcgtt-3′; SEQ ID NO: 1), a single stranded RNA, imidazoquinolines and analogs thereof, nucleosides and analogs thereof, and a combination thereof. In one example, the nucleotide may include polyinosinic: polycytidylic acid (Poly I:C).

In one example, the method as disclosed herein may further comprise the step of administering an activator compound such as, but is not limited to, Toll-like Receptor-1 (TLR-1) agonist, TLR-2 agonist, TLR-3 agonist, TLR-4 agonist, TLR-5 agonist, TLR-6 agonist, TLR-7 agonist, TLR-8 agonist, TLR-9 agonist, TLR-10 agonist, CD-40L agonist, interferon-α (IFN-α) agonist, IFN-β agonist, IFN-γ agonist, PAMPS agonist, DAMPS agonist, an ALUM (AB(SO₄)₂.12H₂O) (A is an element selected from Na and K; B is an element selected from Al and Cr), such as KA1(SO₄)₂.12H₂O, or a combination thereof.

In another example, the composition as disclosed herein may further comprise an activator compound such as, but is not limited to, Toll-like Receptor-1 (TLR-1) agonist, TLR- 2 agonist, TLR-3 agonist, TLR-4 agonist, TLR-5 agonist, TLR-6 agonist, TLR-7 agonist, TLR-8 agonist, TLR-9 agonist, TLR-10 agonist, CD-40L agonist, interferon-α (IFN-α) agonist, IFN-β agonist, IFN-γ agonist, PAMPS agonist, DAMPS agonist, an ALUM (AB(SO₄)₂.12H₂O) (A is an element selected from Na and K; B is an element selected from Al and Cr), such as KA1(SO₄)₂.12H₂O, and a combination thereof.

Toll-like Receptors (TLR) are a type of pattern recognition receptor (PRR) and recognize molecules that are broadly shared by pathogens but distinguishable from host molecules. In one example, TLR-1 agonists may include, but are not limited to, synthetic triacylated lipoprotein (C₈₁H₁₅₆N₁₀O₁₃S). In one example, a TLR-2 agonist may include, but is not limited to, one or more of a lipoprotein, a peptidoglycan, a bacterial lipopeptide from Mycobacterium tuberculosis, Borrelia Burgdorferi, Treponema pallidum; peptidoglycans from species including Staphylococcus aureus, lipoteichoic acids, mannuronic acids, Neisseria porins, bacterial fimbriae, Yersinia

virulence factors, CMV virions, measles haemagglutinin, zymosan from yeast or heat killed Listeria monocytogenes. In one example, a TLR-3 agonist may include, but is not limited to, Polyinosinic-polycytidylic acid (poly(I:C)), which is a synthetic analog of double-stranded RNA (dsRNA), a molecular pattern associated with viral infection. In one example, a TLR-4 agonist may include, but is not limited to, a lipopolysaccharide, a non-toxic derivative of lipid A such as monophosphoryl lipid A or more particularly 3-Deacylated monophosphoryl lipid A (3D-MPL). In one example, a TLR-5 agonist may include, but is not limited to, flagellin or may be a fragment of flagellin which retains TLR-5 agonist activity. The flagellin can include a polypeptide, but is not limited to, Helicobacter pylori, Salmonella typhimurium, Vibrio cholerae, Serratia marcescens, Shigella flexneri, Treponema pallidum, Legionella pneumophila, Borrelia burgdorferi; Clostridium difficile, Rhirobium meliloti, Agrobacterium tumefaciens; Rhizobium lupine; Bartonella clarridgeiae, Proteus mirabilis, Bacillus subtilis, Listeria monocytogenes, Pseudomonas aeruginosa or Escherichia coli. In one example, a TLR-6 agonist may include, but is not limited to, synthetic diacylated lipoprotein, which may be a synthetic lipoprotein derived from Mycoplasma salivarium similar to MALP-2, a Mycoplasma fermentans derived lipopeptide (LP). In one example, a TLR-7 agonist may include, but is not limited to, single stranded RNA (snRNA), loxoribine, a guanosine analogue at positions N7 and 08, or an imidazoquinoline compound, imiquimod (an imidazoquinoline amine analog to guanosine) or derivative thereof. In one example, a TLR-8 agonist may include, but is not limited to, single stranded RNA (ssRNA) or an imidazoquinoline molecule with anti-viral activity, for example resiquimod (R848); resiquimod. In one example, a TLR-9 agonist may include, but is not limited to, type B CpG oligonucleotide, which are synthetic oligonucleotides that contain unmethylated CpG dinucleotides in particular sequence contexts (CpG motifs). In one example, a TLR-10 agonist may include, but is not limited to, triacylated lipopeptides and a wide variety of other microbial-derived agonists shared by TLR1.

In one example, the immunomodulator may comprise lipopolysaccharide and CD40L, which effect on the immune response is illustrated in the Experimental Section, for example illustrated in FIG. 6.

In one example, the antigen-presenting cell precursor may include, but is not limited to, antigen-presenting cell precursors circulating in the peripheral system of the mammal, antigen-presenting cell precursors present in tissues of the mammal, and combinations thereof. In one example, the antigen-presenting cell precursor may include, but is not limited to, a monocyte, a macrophage and tissue resident lineages thereof. In one example, the antigen-presenting cell precursor may be a monocyte. In one example, the monocvte is a CD14+ monocyte.

In one example, the antigen-presenting cell precursor present in tissues of the mammal may include, but is not limited to liver Kupffer cells and endothelial cells. Liver Kupffer cells are also known as Browicz-Kupffer cells and stellate macrophages, which are specialized macrophages located in the liver lining the walls of the sinusoids that form part of the reticuloendothelial system (RES) (also called mononuclear phagocyte system).

In one example, the antigen-presenting cell precursor may not include an antigen-presenting cell precursor such as, but is not limited to, B cell myeloid dendritic cell, CD141 dendritic cell, and CD123 plasmacytoid dendritic cell.

In one example, the method or composition as described herein induces differentiation of the antigen-presenting cell precursor into dendritic cell. In one example, the immune response may comprise the activation of T cell function. In one example, the immune response may comprise the activation of T cells such as, but is not limited to, CD8+ T cell, CD4+ T cell, NK T Cells and a combination thereof.

In one example, the method or composition is for eliciting an immune response to an antigen present endogenously in a mammal suffering from a chronic disease. The chronic disease includes, but is not limited to, Hepatitis B virus (HBV) infection, Hepatitis C virus (HCV) infection, Human Immunodeficiency Virus (HIV) infection, Epstein-Bar virus (EBV) infection, human Cytomegalovirus infection, Herpes Simplex virus (HSV) infection, Measles virus infection, Rabies virus infection, malaria infection, Helminth infection, or a fungal infection.

As used herein, the term “antigen” refers to an agent that produces an immune response. The antigen may be one or more proteins, polysaccharides, peptides, nucleic acids, protein-polysaccharide conjugates, molecules or haptens that are capable of raising an immune response in a human or animal. In one example, the antigen of interest in the present disclosure includes, but is not limited to, an antigen circulating in the peripheral system of said mammal, an antigen present in a tissue of said mammal, and a combination thereof. Alternatively the antigen may be a whole pathogen, for example an attenuated or inactivated pathogen. The whole inactivated pathogen may further be split, for example a split influenza virus. In one example, the antigen of the present disclosure includes, but is not limited to, a virus, a parasite, helminths, a fungi, a microorganism, an allergen, a tumour cell, and components thereof. In one example, the antigen of the present disclosure includes, but is not limited to, Hepatitis B virus (HBV), Hepatitis C virus (HCV), Human Immunodeficiency Virus (HIV), Epstein-Bar virus (EBV), human Cytomegalovirus, Herpes Simplex virus (HSV), Measles virus, Rabies virus, and components thereof. In one example, the antigen includes, but is not limited to, Hepatitis B surface antigen (HBsAg), Hepatitis B core antigen (HBcAg), Hepatitis B e-antigen (HBeAg) or Hepatitis B polymerase antigen (HBpAg).

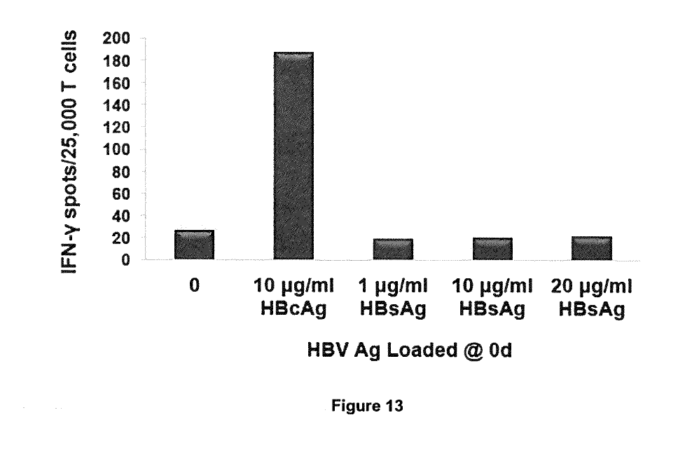

In one example, the antigen of the present disclosure may include, but is not limited to, Hepatitis B surface antigen (HBsAg), Hepatitis B core antigen (HBcAg), Hepatitis B e-antigen (HBeAg) or Hepatitis B polymerase antigen (HBpAg). It is known in the art that HBsAg particles are highly immunogenic and dendritic cells and macrophages from mice cross-present recombinant HBsAg particles to CD8 T cells in the absence of inflammatory signals. HBsAg-specific B cells can present antigen captured through the B cell receptor via the MHC-I pathway. The core antigen (HBcAg) has been shown to bind membrane Ig on a high frequency of resting B cells and activate CD8 T cells. In mice or in vitro model systems, it has been previously demonstrated that HBV antigens have the ability to activate HBV-specific CD8 T cells, which play a key role in HBV control. Additionally, the inventors of the present disclosure found that antigen presenting cells (APCs) are capable of internalizing antigen in the circulation of patients. Furthermore, the inventors of the present disclosure also found that naturally sequestered antigen can be presented to activate virus-specific CD8 T cells in humans. As illustrated in the Experimental Section, FIG. 13, the method or composition of the present disclosure may be used to elicit immune response to various HBV antigens.

In one example, the mammal as used herein refers to a human, monkey, horse or cows. In one example, the mammal is a human.

In one example, the amount sufficient for inducing cell differentiation and/or antigen-presenting function of the antigen-presenting cell precursor that has taken up the antigen includes, but is not limited to, about 1 ng/mL to about 100 μg/mL, about 50 ng/mL to about 100 μg/mL, about 50 ng/mL to about 50 μg/mL, about 50 ng/mL to about 10 μg/mL, about 50 ng/mL to about 1 μg/mL, about 100 ng/mL to about 100 μg/mL, about 100 ng/mL to about 50 μg/mL, about 100 ng/mL to about 10 μg/mL, about 100 ng/mL to about 1 μg/mL, about 500 ng/mL to about 100 μg/mL, about 500 ng/mL to about 50 μg/mL, about 500 ng/mL to about 10 μg/mL, or about 500 ng/mL to about 1 μg/mL.

In one example, the amount sufficient for inducing cell differentiation and/or antigen-presenting function of the antigen-presenting cell precursor that has taken up the antigen may be an amount sufficient to reach a concentration equivalent to between about 1 ng/ml to about 100 ng/ml, or between about 2 to 50 ng/ml, or between about 3 to 10 ng/ml, or between about 4 to 20 ng/ml, or about 3 ng/ml, or about 4 ng/ml, or about 5 ng/ml, or about 6 ng/ml, about 7 ng/ml, about 8 ng/ml, about 9 ng/ml or about 10 ng/ml in vivo.

In another aspect, there is provided an adjuvant comprising the composition as described herein. As used herein, the term “adjuvant” refers to the composition comprising the composition of the present disclosure which assists in inducing an immune response to the antigen. In one example, the adjuvant of the present disclosure elicits or enhances immune response to the antigen.

Also disclosed is a kit comprising the above mentioned adjuvant composition. In one example, the kit may comprise the vaccine as described herein. In one example, the kit may comprise a vaccine comprising the adjuvant as described herein and an antigen-presenting cell precursor that has taken up endogenous antigen. In one example, the kit may be substantially free of exogenous antigen.

In another aspect, there is provided the use of at least one immunomodulator in the manufacture of a medicament for eliciting an immune response to an antigen present endogenously in mammal. The immunomodulator as described herein may be provided in an amount sufficient for inducing cell differentiation and/or antigen-presenting function of an antigen-presenting cell precursor. The antigen-presenting cell precursor may have taken up the antigen.

In another aspect, there is provided a vaccine comprising the composition of the present disclosure and an antigen-presenting cell precursor that has taken up endogenous antigen.

In another aspect, there is provided an immunomodulator that may be capable of eliciting an immune response to an antigen present endogenously in a mammal. The immunomodulator may be present in an amount sufficient for inducing cell differentiation and/or antigen-presenting cell precursor. The antigen-presenting cell precursor may have taken up the antigen.

In one example, there is provided a method of improving or enhancing the immune response of patients that has chronic HBV infection. The method comprises administering immunomodulators as described herein into the human patients. The method as described herein would then act as adjuvant that triggers the differentiation of antigen-presenting cell precursor that preferably has taken up HBV antigen. The differentiation of the antigen-presenting cell precursor by immunomodulator then improves the presentation of HBV antigen to T cells, thereby activating HBV specific T cells. The activated T cells may be CD4, CD8 or NK T cells.

In yet another aspect, there is provided a pharmaceutical composition comprising at least one peptide as described herein. In one aspect, the present disclosure provides a method of administering any of the compositions described herein to a subject. When administered, the compositions are applied in a therapeutically effective, pharmaceutically acceptable amount as a pharmaceutically acceptable formulation. As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. The dose to the subject may be such that a therapeutically effective amount of one or more active compounds reaches the active site(s) within the subject. A “therapeutically effective” or an “effective” dose, as used herein, means that amount necessary to delay the onset of inhibit the progression of, halt altogether the onset or progression of, diagnose a particular condition being treated, or otherwise achieve a medically desirable result, i.e., that amount which is capable of at least partially preventing, reversing, reducing, decreasing, ameliorating, or otherwise suppressing the particular condition being treated. A therapeutically effective amount can be determined on an individual basis and will be based, at least in part, on consideration of the species of mammal, the mammal's age, sex, size, and health; the composition used, the type of delivery system used; the time of administration relative to the severity of the disease; and whether a single, multiple, or controlled-release dose regiment is employed. A therapeutically effective amount can be determined by one of ordinary skill in the art employing, such factors and using no more than routine experimentation.

In administering the systems and methods of the invention to a subject, dosing amounts, dosing schedules, routes of administration, and the like may be selected so as to affect known activities of these systems and methods. Dosage may be adjusted appropriately to achieve desired drug levels, local or systemic, depending upon the mode of administration. The doses may be given in one or several administrations per day.

Administration of a composition of the invention to a subject may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the active compound(s) of the composition within the subject without causing clinically unacceptable adverse effects. The administration may be localized (i.e., to a particular region, physiological system, tissue, organ, or cell type) or systemic, depending on the condition being treated. For example, the composition may be administered orally, vaginally, rectally, buccally, pulmonary, topically, nasally, transdermally, through parenteral injection or implantation, via surgical administration, or any other method of administration where suitable access to a target is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred in sonic embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the composition. Other oral compositions suitable for use with the invention include solutions or suspensions in aqueous or non-aqueous liquids such as a syrup, an elixir, or an emulsion. In one example, the composition may be used to fortify a food or a beverage. Injections can be e.g., intravenous, intradermal, subcutaneous, intramuscular, or interperitoneal. The systems and methods as described herein can be administered by any method which allows the composition of the invention to reach the target cells, e.g., tumour cells. These methods include, e.g., injection, infusion, deposition, implantation, anal or vaginal supposition, oral ingestion, inhalation, topical administration, etc.

In one example, the adjuvant composition may be administered parenterally. In one example, parenteral administration includes, but is not limited to, intramuscular, subcutaneous, subdermal or intradermal administration. In one example, suitable devices for parenteral administration include, but is not limited to, needle (including microneedle) injectors, or transdermal delivery systems.

The parenteral formulation may readily be prepared by someone skilled in the art according to standard methods. In one example, the parenteral formulation may be prepared as an oil in water emulsion. The preparation of parenteral formulations under sterile conditions, for example, by filtration may readily be accomplished using standard pharmaceutical techniques well known to those skilled in the art.

The adjuvant composition according to the present disclosure may be administered to humans and many different target animals, such as for example pigs, cattle, poultry, dogs, cats, horses and the like.

Other delivery systems suitable for use with the present disclosure include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters. polyhydroxybutyric acid, and/or combinations of these.

In one example, a composition may include a suitable pharmaceutically acceptable carrier, for example, as incorporated into a liposome, incorporated into a polymer release system, or suspended in a liquid, e.g., in a dissolved form or a colloidal form, such as in a colloidal dispersion system. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the present disclosure are combined to facilitate the application of the composition. The carrier may be co-mingled or otherwise mixed with one or more active compounds of the present disclosure, and with each other. In a manner such that there is no interaction which would substantially impair the desired pharmaceutical efficacy. The carrier may be either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In one example, the compositions of the present disclosure may include pharmaceutically acceptable carriers with formulation. Ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or *algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens or thimerosal. Suitable carrier concentrations can be determined by those of ordinary skill in the art, using no more than routine experimentation. The compositions of the present disclosure may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain examples. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishes, electrolyte replenishes (such as those based on Ringer's dextrose), and the like. Preservatives and other additives may also be present such as, for example, antimicrobials, antioxidants, chelating agents and inert gases and the like. In addition, sterile, fixed oils are conventionally employed as a solvent or suspending-Medium. For this purpose, any bland fixed oil may be employed including synthetic mono- or di-glycerides. In addition, fatty acids such as oleic acid may be used in the preparation of injectables.

In some examples, the compositions of the present disclosure may be present as pharmaceutically acceptable salts. The term “pharmaceutically acceptable salts” includes salts of the composition, prepared in combination with, for example, acids or bases, depending on the particular compounds found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

### Experimental Section

### Materials and Methods

### Patient PBMC

Blood was collected from 57 chronic HBV patients under informed consent at The Barts and The London Hospital in London, UK and Azienda Ospedaliero-Universitaria di Parma, Italy and the National University Hospital in Singapore. PBMC were isolated by ficoll density gradient separation and cryopreserved in liquid nitrogen. PBMC from healthy donors were obtained from the Blood donation center at National University Hospital of Singapore. All patients used in antigen-specific T cell assays were confirmed to be HLA-A0201+ and HLA-Cw0801+ by molecular based HLA typing.

### FACs Sorting

PBMC were stained with HLA-DR-Alexa700, CD3-FITC, CD7-FITC, CD56-FITC (eBioscience); CD14-PeCy7, CD16-APC-H7, CD20-Horizon V450, CD11c-APC (BD Biosciences); CD141-PE (BDCA3; Miltenvi Bitotech); CD123 PerCp-Cy5.5 (Biolegend) for 30 min at 4 C. Viability dyes, Dapi or Live/Dead yellow fixable stain (Invitrogen), were used to gate on live cells followed by singlet gates. Cells were sorted on BD FACs Aria III to greater than 98% purity. Data generated from the sorting files was used to calculate the frequency of APC populations in all subjects.

### Mixed Lymphocyte Reactions

Sorted APC populations from healthy donors and chronic HBV patients were co-cultured with cryopreserved total T cells isolated from a single healthy donor using RosetteSep Human T Cell Enrichment Cocktail (Stemcell Technologies). T cells were labeled with CFSE (Invitrogen) and added at 20:1 effector:target ratio (100,000 T cells:5,000 APCs) to sorted APCs and cultured for 7 d in AIM-V medium supplemented with 2% human AB serum (Invitrogen). T cells alone served as negative control and T cells plus anti-CD3/anti-CD28 beads served as positive control. After 7 d, cells were stained with anti-CD3-Horizon V450 (BD bioscences) and acquired on a BD LSR-II flow cytometer. Resulting data was then analyzed using the FlowJo Proliferation Platform to determine the division index and proliferation index of each APC population from healthy donors and chronic HBV patients. The division Index is the average number of cell divisions that a cell in the original population has undergone. This is an average even for cells that never divided. The proliferation index was used to compare between patients and is defined as the average number of cell divisions that the responding cells underwent.

### HBsAg Staining

APCs were sorted from chronic HBV patients as described above for fluorescent microscopy analysis. Sorted APCs were fixed with 1% paraformaldehyde for 15 min at room temperature. Fixed cells were then blocked with 0.25% saponin in PBS, 2% BSA 5% goat serum (Serotec) for a minimum of 30 minutes and stained with polyclonal anti-HBsAg-biotin (4.5 μg/ml; Ad/Ay; Abeam) for 1 h at room temperature. Cells were washed two times with PBS/1%BSA/0.1% saponin and then stained with streptavidin-FITC or streptavidin-APC (BD Biosciences) for 30 min at room temperature. After two washes cells were loaded onto superfrost plus adhesion or polysine slides (Thermo) and mounted with Prolong Gold antifade+Dapi (Invitrogen), sealed and acquired on a TissueFAXs system (TissueGnostics). The TissueFAXs system collects a digital image of the cytospin cells and calculates the intensity of FITC (HBsAg) fluorescence on DAPI positive cells to give quantitative data on the frequency of HBsAg+ cells in the APC population. FITC was chosen as the readout channel because this was used to gate out lineage CD3, CD7 and CD56 markers. Therefore, there were no FITC positive markers on the APC populations. CD14 MNs were also sorted by single color staining using PeCy7 CD14 and isolated using CD14 microbeads to confirm that HBsAg staining was specific and not an artifact of the 9 color sorting panel. The relatively low quantity of HBsAg required a two-step biotin-streptavidin staining and long exposure times (250-350 ms). As a result, autofluorescence and background staining was higher in CD14 MN and reached up to 15% in some healthy donors. TissueQuest 3.0 was used to analyze fluorescent microscopy data with a minimum of 25 fields of view analyzed for each cell population to obtain sufficient numbers for analysis.

### Ex Vivo Antigen Presentation Assays

Sorted APC populations; CD11c DC, CD141 DC, CD123 pDC, CD14 MN, CD16 MN and CD20 B cells were cultured overnight in media alone or under activating conditions +/−10 μ/ml recombinant HBsAg (Kindly provided by DynaVax). Cells were activated with 10 μg/ml polyI:C (TLR-3), 5 μM CpG ODN2216 (TLR-9), 1 μg/ml LPS (TLR-4), 5 μg/ml Imiquimod (TLR-7), 1 μg/ml ssRNA40 (TLR-8) (InvivoGen), 1000 U/ml IFN-α or 1000 U/ml IFN-γ (R&D systems). Following overnight incubation, APC populations were thoroughly washed and transferred to anti-IFN-γ coated Elispot plates and co-cultured with CD8 s183-TCR or s171-TCR redirected T cells at 5:1 E:T ratio (normally 5,000 APCs/well but as low as 1000 CD141 DC/well) for 24 h. APCs pulsed with 5 μg/ml HBs183-91(FLLTRILTI), HBs171-80 (FLGPLLVLQA) or HBc18-27 (FLPSDFFPSV) peptide served as positive control for T cell activation. IFN-γ Elispot was performed as described previously in Tan et al 2008, using 5 μg/ml of 1-DIK for capture and 0.5 μg/ml 7B6-1-Biotin plus Streptavidin-ALP for detection (Mabtec) (Tan, A. T, Loggi, E., Boni, C., Chia, A. Gehring, A. J., Sastry, K. S., Goh, V., Fisicaro, P., Andreone, P., Brander, C., et al. 2008. Host ethnicity and virus genotype shape the hepatitis B virus-specific T-cell repertoire. J. Virol 82: 10986-10997). Elispot plates were developed and analyzed using CTL Immunospot analyzer. Cutoff values for positive responses were set according to cumulative data from healthy donors and HLA mismatched chronic HBV responses (data not shown). Wells were considered positive if responses were above 20 spot forming units/50,000 T cells.

### Monocyte-Derived DC Generation and Cross-Presentation

CD14+ MN were isolated from healthy donors and chronic HBV patients by FACs sorting or positive selection using CD14 micobeads (greater than 90% purity; Miltenyi Biothech). Purified monocytes were then cultured in RPMI 1640 supplemented with 10% heat-inactivated FBS, 20 mM HEPES, 0.5 mM sodium pyruvate. 100 U/ml penicillin, 100 μg/ml streptomycin, MeM amino acids, Glutamax, MeM nonessential amino acids, 55 μM 2-mercaptoethanol (Invitrogen). GM-CSF+IL-4 moDC were made by culturing CD14 MN in 50 ng/ml (1500 U/ml) IL-4 and 50 ng/ml (750 U/ml) GM-CSF (R&D systems) for 6 d. GM-CSF+IL-15 (R&D Systems) moDC were made by culturing CD14 MN in 100 ng/ml (1500 U/ml) GM-CSF+200 ng/ml IL-15. Half of the media was removed after 3 d and replaced with complete RPMI containing cytokines. After 6 d of incubation, moDC were stained with CD14-PerCp (Miltenyi Biotech), HLA-DR alexa700 (eBioscience), CD80-FITC (BD Bioscience) and CD86-APC (Biolegend) to confirm differentiation.

Six day moDC were cultured overnight in media alone or activated with 10 μg/ml polyI:C or 0.1 ug/ml LPS (Invivogen)+50 ng/ml soluble HEK-derived CD40L (R&D Systems). Following overnight incubation, moDC were washed three times with HBSS and co-cultured at 2:1 effector:target ratio (10,000 T cells:5000 moDC) with CD8 HBs183-91_{T} or HBs171-80-specific TCR redirected T cells for IFN-γ elispot. A cutoff for positive response, 25 IFN-γ spots/25,000 T cells for non-activated moDC and 35 IFN-γ spots/25000 T cells for activated moDC, was set based on the cumulative data of negative controls from healthy donors and HLA-mismatched chronic HBV patients (FIGS. 5 & 6).

To test cross-presentation from healthy donors, CD14 MN were loaded with HBV antigen at day zero (HBsAg 1-20 μg/ml) for 4 h in complete RPMI and then differentiated to moDC. Following the loading incubation at 0 d, 50,000 CD14 MN were stained for HBsAg to confirm uptake.

Autologous T Cell Expansion With moDC

Patients PBMC were expanded with synthetic 15mer overlapping peptides as previously described (Tan et al 2008, as cited above). Briefly, 20% of PBMC were loaded with 10 μg/ml peptide pools covering the whole HBcAg and HBsAg and incubated for 1 h at 37′C. Peptide loaded cells were washed and mixed back with remaining PBMC and expanded in vitro for 10 d in Aim-V+2% human AB serum+20 U/ml IL-2 (R&D Systems). After 10 d expansion cells were stimulated with overlapping peptide pools and HBV-specific T cells were quantified using IFN-γ elispot. Positive responses were defined as two times the mean of unstimulated wells.

For autologous moDC expansion, CD14 MN were isolated and differentiated for 6 d in the conditions described above. On day 6, moDC were activated with LPS and CD40L for 24 h and then mixed with autologous PBMC at 1:10 (moDC:PBMC) ratio. Cells were incubated for 10 d in Aim-V 2% human AB serum+20 U/ml IL-2. After 10 d, T cell responses were tested using IFN-γ elispot with overlapping peptides. Positive cutoff for moDC expanded T cells was two times the mean of unstimulated wells or greater than 10 spots. When cell numbers permitted, positive responses to moDC expansion were tested by intracellular cytokine staining for IFN-γ to confirm elispot responses.

### TCR-Redirected T Cell Function

T cells from healthy donors were transduced with retroviral vectors carrying TCRs specific for the HLA-A2 restricted HBV surface (HBs183-91; FLLTRILTI) and core (HBc18-27; FLPSDFFPSV) antigen or HLA-Cw0801 restricted HBs171-80 epitope (FLGPLLVLQA) as previously described with 18 μg MP71-TCR together with 6 μg of amphotropic envelope.

To examine their cytokine profile, s183-TCR transduced T cells were stimulated with HLA-A2+ T2 cells loaded with 1 μg/ml of the respective peptide and incubated overnight at 37 C with 2 μg/ml brefeldin A (Sigma). T cells were labelled with viability dye and stained with CD3-biotin+streptavidin-Q605, CD4-Q655 (Invitrogen) and CD8-Horizon V500 and then fixed with Cytofix/Cytoperm (BD biosciences). T cells were stained with IFN-γ-Horizon V450, IL-4-Fitc, IL-2-PerCp-Cy5. IL-2-PE, TNF-α-PE-Cy7, IL-10-APC, IL-17a-Horizon V450, CXCL-8-PE, Mip1β-PeCy7 (BD Biosciences), Mip1α (R&D systems) and GM-CSF-Alexa647 (eBioscience). Samples were collected on BD LSR-II and analyzed using FACs Diva software. Transduced T cell populations used to readout antigen presentation assays ranged from 30-50% IFN-γ+CD8+, which showed no significant difference in sensitivity compared to T cell clones (data not shown)

### Constitutive Antigen Presentation Assays

APC populations were sorted from 7 HLA-A2+ chronic HBV patients or healthy donors to greater than 98% purity. TCR transduced T cells were then cultured at 5:1 effector:target ratio with each APC population (typically 5,000 APC/well for Elispot and 10,000 APC/well for intracellular cytokine staining) and T cell activation was monitored using IFN-γ intracellular cytokine staining or IFN-γ Elispot. For intracellular cytokine staining, cells were co-cultured overnight in V-bottom 96 well plates in 2 μg/ml brefeldin A. Cells were labeled with viability dye, stained with CD8-Horizon V500 (BD Biosciences) and fixed in cytofix/cytoperm (BD Biosciences). Cells were then stained with IFN-γ-Horizon V450 (BD Biosciences) to monitor T cell activation. As positive control, CD14 MN were pulsed with 5 μg/ml HBs183-91 or HBc18-27 peptide for 1 h and washed. IFN-γ Elispot was performed as described previously using 5 μg/ml of 1-DK1 for capture and 0.5 μg/ml 7B6-1-Biotin plus Streptavidin-ALP for detection (Mabtec). Wells were considered positive if responses were above 20 spot forming units/25,000 T cells. An HLA-A2+ healthy donor was included in each experiment to determine the background for each experiment. HLA-A2− donors were also tested to confirm specificity and restriction of the transduced T cells (data not shown).

### Results

### Professional APC Frequency and Function in Chronic HBV Patients

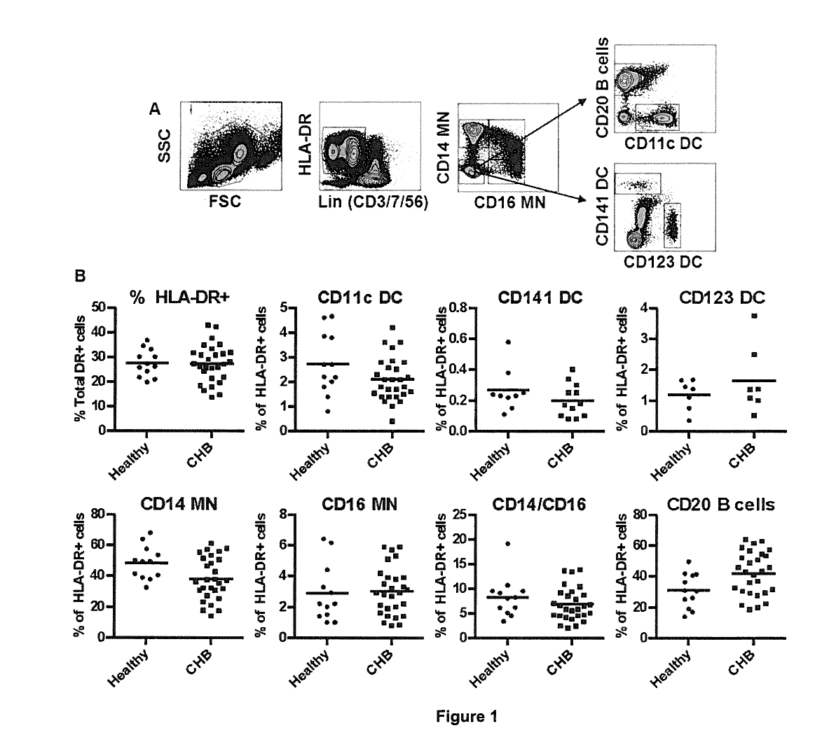

Controversy exists in chronic HBV infection whether the frequency and function of APCs is intact. Therefore, before investigating questions related to antigen-specific T cell activation in the circulation, APC compartment in 28 chronic HBV patients were characterised (Table 1). Analysis of the frequency of total APCs (HLA-DR+) or seven distinct APC populations ex vivo (FIG. 1A; myeloid dendritic cells (mDC), CD141 DC, CD123 plasmacytoid DC, CD14 monocytes (CD14 MN), CD14/CD16 MN, CD16+/CD14 low MN (CD16 MN), and CD20 B cells) did not show any significant differences between chronic HBV patients and healthy controls (FIG. 1B). However, the frequency of CD14 MN was found to appear to be lower in chronic HBV patients while the frequency of CD20 B cells tended to be increased but these differences were not significant.

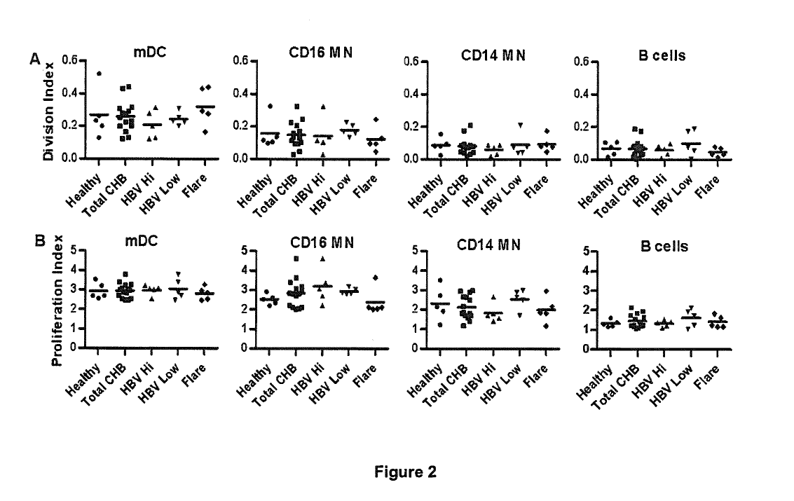

The stimulatory capacity of four of the APC populations were tested using allogeneic mixed lymphocyte reactions (MLR) where sufficient numbers could be consistently obtained. The division index and proliferation index were calculated for T cells after co-culture with sorted APCs. Similar to their frequency, no significant differences were observed in the ability of professional APCs from chronic HBV patients (Table 2) to stimulate proliferation compared to healthy donors (FIG. 2). This suggests that at least these four APC populations from chronic HBV patients are not inherently inhibitory.

### HBV Surface Antigen in Ex Vivo Isolated APCs

Having shown that the frequency and function of APCs was generally intact in the cohort of chronic HBV patients, the capability of circulating HBV antigen to be internalized by APCs in the blood was investigated. Using PCR and electron microscopy, HBV antigen has been shown to be present in in dendritic cells. However, due to the sensitivity of these assays and limited quantitative data from electron microscopy, controversy remains as to whether it is accurate or due to trace contamination with serum or cells. To clarify these issue six APC populations were sorted and stained for HBV surface antigen (HBsAg). The sorted populations were analyzed the cells using the TissueFax system, as it allows for quantitative analysis of fluorescent microscopy data.

Only CD14 MN in the circulation of chronic patients stained positive for HBsAg; CD16 MN, mDC, CD141 DC, pDC and B cells were negative, with background similar to what is observed in healthy controls (FIGS. 3A & B). CD14 MN were positive for HBsAg in 12 out of 19 patients tested (Table 3). The frequency of HBsAg- CD14 MN ranged from 20 -96% with a mean of 43% (FIG. 3B). Similar to the representative patient shown, CD16 MN, mDC, CD141 DC, pDC and B cells were negative for HBsAg after screening multiple patients (FIG. 3D). Thus, CD14 MNs are the only cell population carrying a detectable depot of viral antigen in the circulation of chronic HBV patients.

Since HBsAg+ CD14 MN were not detectable in all patients (n=7), the possibility that HBsAg staining in CD14 MN may correlate with viral parameters (HBV DNA and HBsAg) was examined. Patients with higher viral load presented higher frequencies of HBsAg+ CD14 MN (FIG. 3C). However, this was inconsistent because patients with low viral load (<10³) showed HBsAg+ CD14 MN similar to those with HBV-DNA levels of 10⁴-10⁵ (FIG. 3C). Furthermore, HBsAg negative CD14 MN were found in patients with HBV DNA similar to those that stained positive (data not shown). This study also observed that a 3 Log 10 drop in viral load did not influence HBsAg staining, all suggesting the HBsAg positivity is not directly related to viremia (FIG. 3D).

This study then analyzed whether only HBsAg levels directly correlate with HBsAg+ staining in CD 14 MN. Five patients were selected under antiviral therapy where HBV DNA values were negative by clinical testing but had increasing levels of HBsAg in the serum. A dose dependent increase in HBsAg+CD14 MN occurred as HBsAg increased in the circulation (FIG. 3E), confirming that detection of HBsAg+ CD14 MN is directly related to the level of circulating viral antigen. Staining became maximal at approximately 3306 IU/ml HBsAg (equivalent ≈18 μg/ml). The lowest concentration tested, 606 IU/ml (≈3.3 μg/ml), was just above background staining (data not shown), indicating that the detection limit for HBsAg staining has a cutoff of roughly 3 μg/ml HBsAg in the serum.

### Testing Ex Vivo MHC-I Presentation by APCs Using HBsAg-Specific Redirected T Cells

Next, it was investigated whether in vivo captured HBsAg can be constitutively presented on HLA-class I to HBV-specific CD8 T cells. TCR gene transfer was used to engineer HBV-specific T cells (FIG. 12; Gehring, A. J., Xue, S. A., Ho, Z. Z., Teoh, D., Ruedl, C., Chia, A., Koh, S., Lim, S. G., Maini, M. K., Stauss, H. et al. 2011. Engineering virus-specific T cells that target HBV infected hepatocytes and hepatocellular carcinoma cell lines. J Hepatol 55: 103-110). Lymphocytes of healthy donors were transduced with HLA-A2 or HLA-Cw08 restricted HBsAg-specific TCRs and used to detect MHC-I antigen presentation in all subsequent functional assays.

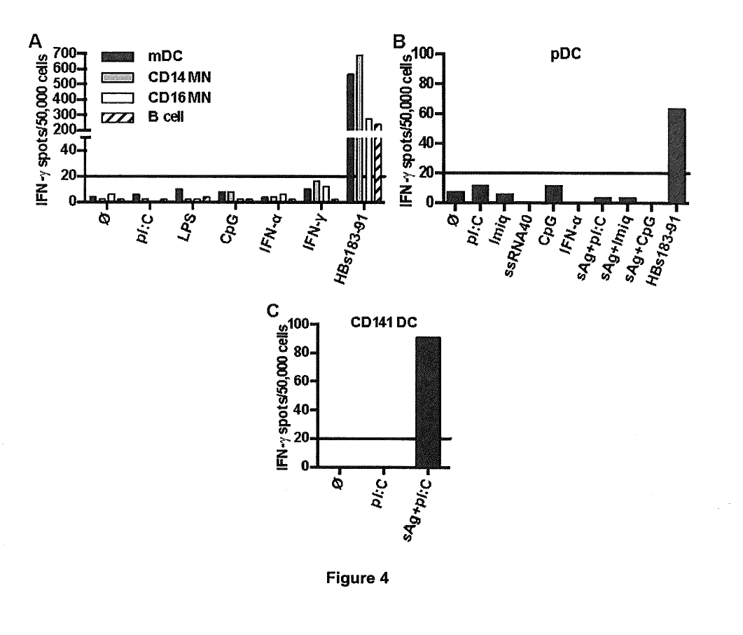

APCs were sorted from 7 chronic HBV patients and 4 healthy donors (Table 4). Purified APCs were co-cultured with appropriately matched, HLA-restricted HBsAg-specific TCR redirected T cells. T cell activation, measured by IFN-γ production, was used to determine if ex vivo APCs isolated from chronic HBV patients presented HBsAg epitopes on MHC-I. Co-culture with APCs of either healthy donors or chronic HBV patients ex vivo did not stimulate any significant T cell IFN-γ production as measured by IFN-γ elispot (FIG. 4, Ø). The negative results were confirmed using flow cytometry to monitor T cell IFN-γ production (data not shown). The study then tested whether inflammatory stimuli (adjuvants) could induce the cross-presentation of in vivo captured antigen by ex vivo isolated APCs. Sorted mDC, CD14 MN, CD16 MN or B cells were activated with TLR-3, -4, -9 ligands, IFN-α or IFN-γ and co-cultured with HBsAg-specific TCR transduced T cells. Similarly, under these conditions, T cell activation was not observed, indicating that even after short-term activation, mDC, CD14 MN, CD16 MN or B cells did not cross-present circulating HBsAg (FIG. 4A).

Three chronic HBV patients with the correct HLA profile had sufficient samples to directly test cross-presentation by CD141 DC & plasmacytoid DC. Plasmacytoid DC (pDC) has been previously shown to cross-present apoptotic debris or lipoproteins following TLR-7 or influenza stimulation. CD141 DC have recently been described as the human equivalent of the murine CD8α subset, capable of efficiently cross-presenting antigen in humans after activation by poly I:C. Neither resting pDC nor pDC activated with a panel of TLR ligands and IFN-α activated HBsAg-specific T cells ex vivo (FIG. 4B). Fewer conditions were tested with CD141 DC due to the limited cell numbers but neither non-activated nor polyI:C activated CD141 DC from chronic HBV patients stimulated CDS T cells directly ex vivo (FIG. 4C). However, CD141 DC, unlike pDC, efficiently cross-presented recombinant HBsAg (sAg) loaded exogenously when stimulated with polyI:C (FIGS. 4B&C).

Thus, no constitutive or inducible ex vivo cross-presentation of HBsAg was detected in the six different APC populations tested. T cells and APCs were functional as peptide-loaded APCs of chronic patients stimulated T cell IFN-γ production at a level similar to those of healthy donors (FIG. 4 & data not shown). Moreover, the ability of CD141 DC to cross-present soluble antigen was not altered by HBV persistence. The possibility of HBV core antigen (HBcAg) presented to HLA-A2 restricted CD8 T cells ex vivo was also investigated by utilizing HBcAg-specific TCR redirected (c18-TCR) T cells. Similar to HBsAg-specific T cells, no T cell activation was observed with APCs from 4 HLA-A0201 chronic HBV patients (data not shown). Therefore, even though the major populations of APCs in the circulation of chronic HBV patients are constantly exposed to viral antigen (HBsAg & HBcAg), they do not constitutively process and present the antigen via the MHC-I pathway to CD8 T cells.

Cross-Presentation of In Vivo Captured HBsAg by Monocyte-Derived DC from Patients

Since CD14 MNs are the predominant HBsAg+ APC population, the possibility that their differentiation to DC could permit cross-presentation of captured HBV antigen was investigated. Initially, healthy donor CD14 MN was loaded with recombinant HBV antigen at day zero (FIG. 5A). The CD14 MN was differentiated to monocyte-derived DC (moDC) with various conditions and cross-presentation was monitored using TCR-redirected CD8 T cells. Incubation under multiple conditions resulted in the cross-presentation of antigen captured on day zero by CD14 MN. Differentiation alone using GM-CSF alone or GM-CSF+IFN-α for 3 d or GM-CSF+IL-4 (classical method), GM-CSF+IL-15, GM-CSF+IFN-γ or IL-32 for 6 d resulted in cross-presentation of HBV antigen to virus-specific CD8 T cells (FIG. 5A). Cross-presentation was enhanced by activating the different types of DC with LPS and CD40 ligand (FIG. 5B). These data show that multiple differentiation pathways can be used to induce CD14 MN to cross-present persistent antigen they acquire from the circulation.

The possibility whether viral antigen naturally captured by CD14 MN from the patient circulation in vivo can be presented to T cells after ex vivo DC differentiation was also tested. Purified CD14 MN from 14 chronic HBV patients (Table 5) were stained for HBsAg ex vivo to determine their antigen content and differentiated to moDC for 6 days in the presence of IL-4 and GM-CSF. Cross-presentation of in vivo captured HBsAg was monitored using TCR-redirected CD8 T cells. MoDC from 5 HLA-A0201+ chronic patients were tested with the HLA-A0201 restricted HBs183-91 epitope specific T cells, while moDCs derived from 4 HLA-Cw0801+ chronic patients were tested with HLA-Cw0801 restricted HBs171-80 epitope specific T cells. MoDC from 5 HLA-mismatched chronic HBV patients and 3 HLA-matched healthy donors served as negative controls.

Ex vivo HBsAg staining of CD14 MN was observed in seven out of nine HLA-matched patients and in all 5 HLA-mismatched patients (FIG. 6A, black bars). Only one patient, CHB-8, showed HBsAg staining following differentiation to moDC (data not shown). Upon differentiation alone, robust T cell activation was detected in 6/9 HLA-matched chronic HBV patients (FIG. 6B, black bars). MoDC from HLA-matched healthy subjects or HLA-mismatched chronic HBV patients did not stimulate any significant T cell activation (FIG. 6B). Activation of moDC resulted in increased T cell responses; with a total of 8/9 moDC derived from HLA-matched chronic HBV patients able to elicit a positive T cell response (FIG. 6C). Activation of HLA-matched healthy or HLA-mismatched moDC from chronic patients did not result in any significant increase in HBV-specific T cell activation.

On the other hand, variability in responses was observed. Patient CHB-6 did not show any HBsAg staining in CD14 MN ex vivo but was capable of activating HBs183-91 specific T cells upon moDC differentiation, suggesting that HBsAg levels of 3 μg/ml and below (limit of the HBsAg staining assay of the present disclosure) can be efficiently cross-presented. Conversely, patient CHB-9, with HBsAg+ CD14 MN was unable to trigger HBV-specific T cells, perhaps due to the presence of an escape mutation in the HBV epitope. In contrast, CHB-8, the one patient with detectable HBsAg in moDC following differentiation, showed the greatest T cell response upon activation with polyI:C. Taken together, these data show that it is possible to differentiate HBsAg+ CD14 MN from the blood of chronic HBV patients and harness the circulating viral antigen to activate virus-specific CD8 T cells. That is, the administration of an immunomodulator to antigen-presenting precursor cells that have taken up antigen (i.e. HBsAg+ CD14 MN) can elicit immune response (i.e. activate virus specific CD8 T cells).

Expansion of Autologous Virus-Specific T Cells Using moDC Presenting In Vivo Captured HBsAg

Lastly, the present study tested whether cross-presentation of in vivo captured antigen by moDC could be used to expand autologous HBV specific T cells in chronic HBV patients. HBV-specific T cells are functionally and numerically impaired in chronic HBV patients but can still be detected in low numbers in young adults, patients under anti-viral therapy or with low HBV-DNA. Twenty patients were tested (Table 6), 16 under antiviral therapy and an additional 4 that were treatment naïve (CHB-29-32). CD14 MNs were isolated from each patient and differentiated them to moDC with GM-CSF+IL-4. The moDC were activated with LPS+CD40L and co-cultured with autologous PBMC for 10 d; relying only on in vivo captured antigen to expand virus-specific T cells. In parallel, PBMC were expanded using a standard approach with 15mer overlapping peptides covering HBsAg and HBcAg to compare responses between the two methods.

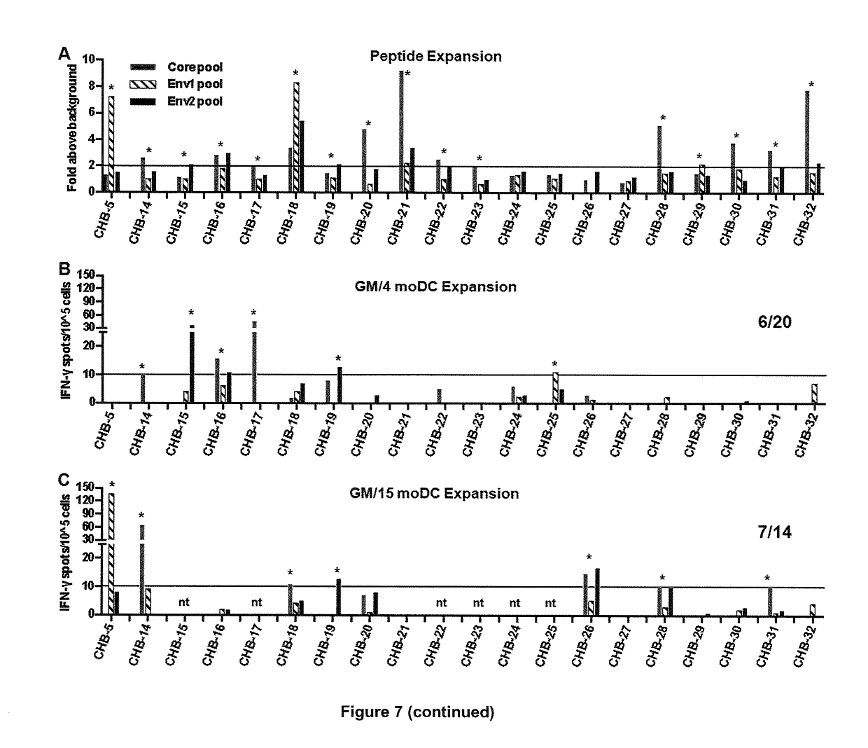

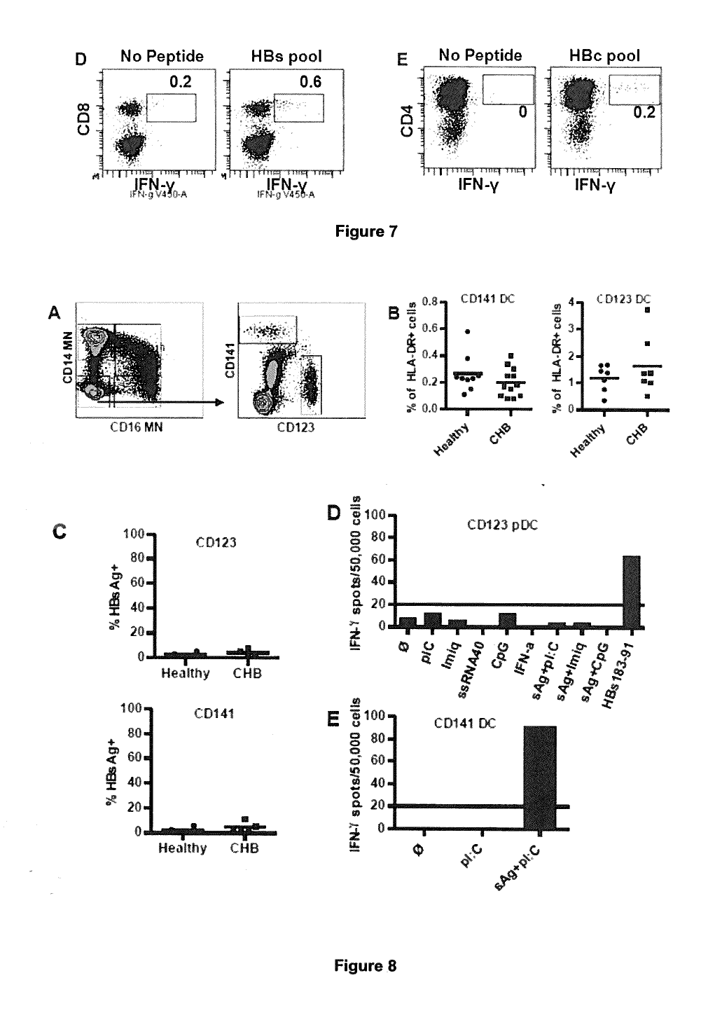

As expected, virus-specific response were negative when assayed directly ex vivo in all patients (data not shown). T cells specific for both HBcAg and HBsAg could instead be expanded in 16 patients after 10 days of in vitro culture with synthetic peptide pools (FIG. 7A). More importantly, PBMC expanded with GM-CSF+IL-4 moDC (4-DC), presenting in vivo captured HBV antigen, resulted in HBV-specific T cell expansion in 6/20 patients (FIG. 7B). Expansion of virus-specific T cells was not only restricted to circulating HBsAg and also resulted in the expansion of HBcAg-specific T cells. No T cell responses to HBV polymerase or X proteins were detected (data not shown). These data suggest that, in addition to HBsAg, either HBeAg, the soluble form of HBcAg, or HBcAg internalized as part of whole virions can be processed and presented to expand virus-specific T cells.

Fewer responses were observed with 4-DC compared to peptides. Boosting virus-specific T cell expansion with 4-DC by blocking PD-L1 and CTLA-4 or inhibiting Bim-mediated apoptosis, two pathways known to increase HBV-specific T cell responses, was ineffective (data not shown). The present study then tested whether the moDC differentiation program would affect virus-specific T cell expansion to determine if A) there is flexibility in how CD14 MN can be induced to cross-present antigen and B) whether different types of moDC might be more efficient at expanding autologous T cells. MoDC made with GM-CSF+IL-15 have been shown to be highly efficient at activating CD8 T cells. Therefore, the ability of 4-DC and GM-CSF+IL-15 (15-DC) moDC to expand virus-specific T cells were compared in 14 CHB patients. FIG. 7C shows that HBV-specific T cells were expanded in 7/14 patients by 15-DC and that 15-DC did enhance T cell responses compared to 4-DC in 6 patients (CHB-5, -14, -18, -26, -28 -31).

The moDC expanded T cells were functionally characterized with intracellular cytokine staining. Monocyte-derived DC presenting the in vivo captured antigen were capable of expanding both CD8 (FIG. 7D) and CD4 (FIG. 7E) T cells. However, these cells displayed an exhausted functional phenotype and produced only IFN-γ but not TNF-α or IL-2 (data not shown).

The specificity of the T cells expanded by DC largely overlapped with that of PBMC expanded with synthetic peptides (FIG. 7A). However, patient CHB-25, -26 & -28 did show an HBV specific T cell response in the absence of any positive responses with peptides. This response could be due to differences between the consensus synthetic peptides and the endogenous antigen presented by moDC. Overall, these data confirm that the depot of viral antigen present in circulating monocytes can serve as a personalized antigenic reservoir to expand autologous virus-specific T cells.

### Role of CD141 and Plasmacytoid DC

Cell numbers are a constant limitation with human research; however, sufficient PBMC from three chronic HBV patients were obtained to isolate six APC populations; the four mentioned above as well as the specialized DC subsets, CD141 DC & plasmacytoid DC. Plasmacytoid DC (pDC) have been shown to cross-present apoptotic debris or lipoproteins following TLR-7 or influenza stimulation and the CD141+DC have recently been described as the murine equivalent CD8α subset, capable of efficiently cross-presenting antigen in humans after activation by poly I:C. The CD141 DC are CD11c low and represent 5-10% of the myeloid DC population (0.05% of total PBMC), which means it is necessary to sort 50-60 million PBMC (60 ml whole blood) from patients to obtain enough for antigen cross-presentation experiments.

The populations were identified from the lineage negative, HLA-DR+, CD14-/CD16-cells (FIG. 8A) and the frequency of CD141 DC and pDC was analyzed in chronic HBV patients and healthy controls (FIG. 8B). The frequency of these rare DC subsets was similar in both groups, showing that HBV persistence does not impact the CD141 DC and pDC populations. FACs sorted CD141 DC and pDC from chronic HBV patients were analyzed by TissueFaxs but no detectable HBsAg was observed ex vivo (FIG. 8C).

The inventors of the present disclosure then tested if these specialized populations of DC were capable of cross-presenting in vivo captured HBsAg either constitutively or after activation. Chronic HBV patient pDC were left untreated or activated with a panel of TLR ligands and IFN-α, but none of these conditions resulted in HBsAg-specific T cell activation (FIG. 8D). Fewer conditions were tested with CD141 DC due to the limited cell numbers but neither non-activated nor polyI:C activated CD141 DC from chronic HBV patients activated CD8 T cells directly ex vivo (FIG. 8E). However, CD141 DC, unlike pDC, efficiently cross-presented recombinant HBsAg (sAg) loaded exogenously when stimulated with polyI:C (FIG. 8E). Thus, constitutive or inducible ex vivo cross-presentation of HBsAg does not occur in the specialized CD141 DC or pDC of chronic HBV patients but the ability of CD141 DC to cross-present soluble antigen is not altered by HBV persistence.

Cross-Presentation of In Vivo Captured HBsAg by Monocyte-Derived DC from Patients

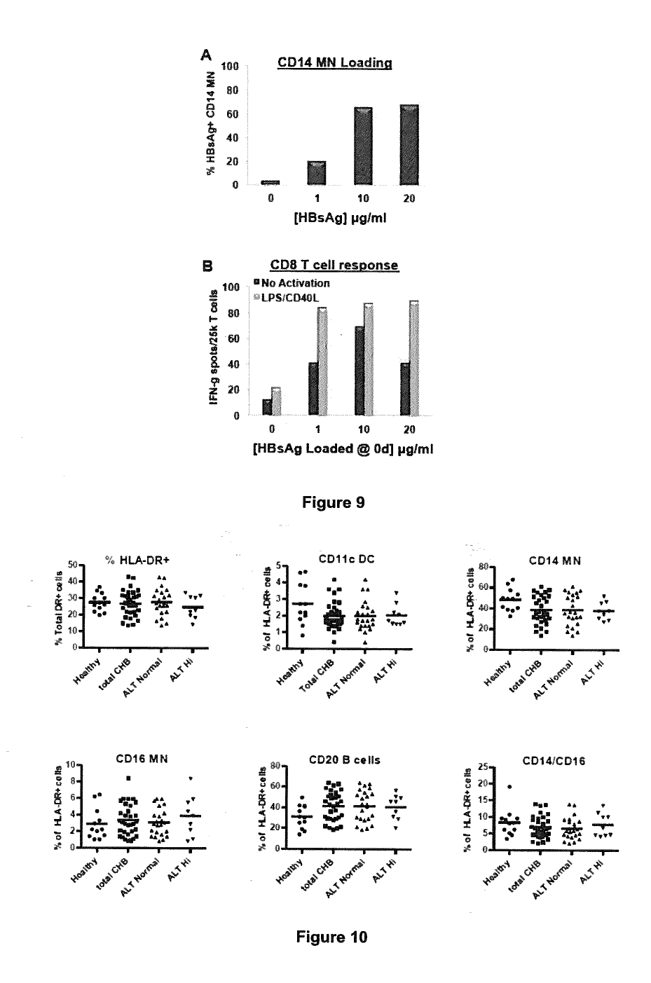

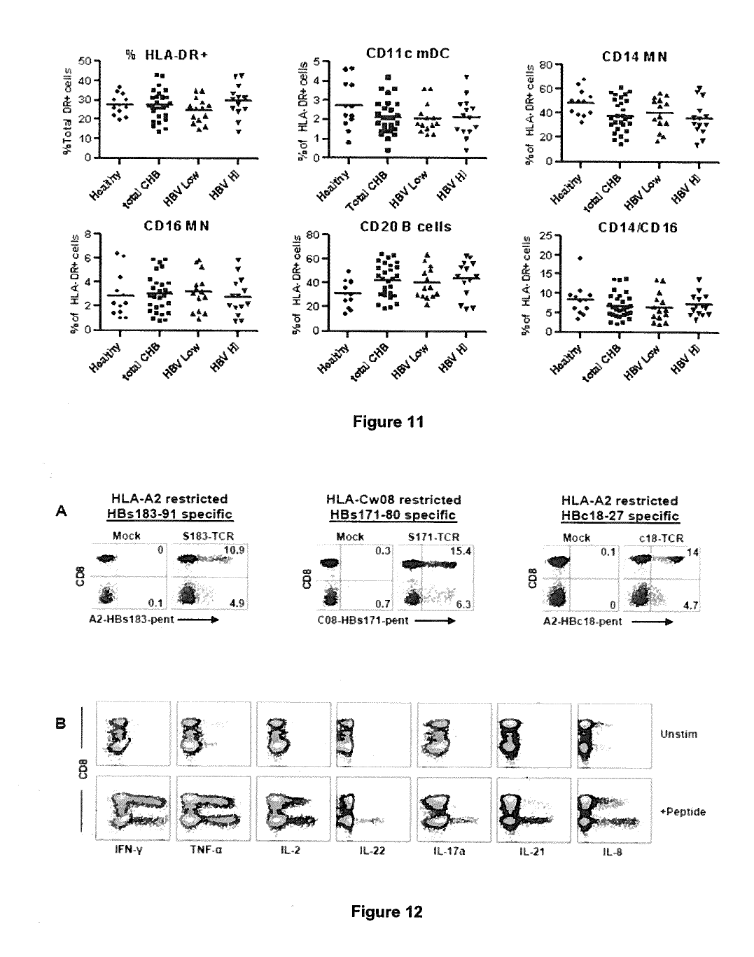

None of the six APC populations isolated ex vivo from chronic HBV patients could cross-present in vivo captured HBsAg. Since CD14 MNs are the predominant HBsAg+ APC population, it may be possible that their differentiation to dendritic cells could permit cross-presentation of captured HBV antigen. This possibility was initially tested by loading healthy donor CD14 MN with increasing amounts of rHBsAg at day zero (FIG. 9A), differentiated them to monocyte-derived DC (moDC) with GM-CSF and IL-4 and monitored cross-presentation using the TCR-redirected CD8 T cells. Differentiation alone resulted in dose dependent cross-presentation of HBsAg to virus-specific CD8 T cells, which could be enhanced with moDC activation (FIG. 9B). The specificity of HBV-specific T cell activation was confirmed using HBc18-27-specific TCR (c18-TCR) redirected cells that did not recognize HBsAg loaded moDC but did respond to HBcAg loaded moDC, (FIG. 13).

The present disclosure determines if persistent antigen present during chronic viral infection could serve as a personalized antigenic reservoir to activate antigen-specific T cells for potential therapeutic intervention. The persistent presence of high quantities of virions and viral antigens in the circulation of patients with chronic hepatitis B has been associated with the immunological alterations present in these patients. Specifically, that persistent exposure to HBV and HBsAg alters the frequency and function of myeloid, plasmacytoid and monocyte-derived DC. The previous reports regarding the HBsAg inhibitory effects were largely related to TLR-mediated cytokine production and could affect innate immunity pathways. However, analyses of the present disclosure of the T cell stimulatory function of different APCs ex vivo did not show any significant alterations. Despite marked heterogeneity between different subjects, no significant quantitative alterations in circulating monocytes, dendritic cells or B cells were observed. The APCs of chronic patients stimulated T cell proliferation in MLR reactions, efficiently activated HBV-specific T cells when loaded with synthetic peptides and preserved, as in the case of CD141 DC, their cross-presentation capacity. Combined with the data demonstrating that cross-presentation could be induced in monocytes upon differentiation to DC suggests that antigen processing and presentation is not affected in chronic HBV patients. Therefore, the circulating viral antigen does not alter the functionality of different APCs. To the contrary, circulating antigen selectively captured by CD14 monocytes can be used to as a personalized antigenic reservoir to activate virus-specific T cells in chronic HBV patients.

Circulating HBsAg was undetectable in 5 out of 6 APC populations tested but was internalized and retained at detectable levels selectively by CD14 MN. Staining in the present disclosure showed that the frequency of HBsAg+ CD14 MN did not change significantly with antiviral therapy, which reduced viral load, but was directly correlated with the amount of circulating HBsAg in chronic patients. However, due to the detection limit of the present assay, the presence of HBsAg+ CD14 MN below 3 μg/ml HBsAg in the serum could not be effectively visualised ex vivo. The visual limit did not reach the sensitivity of the present T cell assays where T cell activation by moDC derived from HBsAg negative CD14 MN from chronic patients were observed, suggesting that naturally captured antigen below the detection limit is also efficiently processed.

Nevertheless, no direct ex vivo evidence for constitutive or inducible cross-presentation in the circulation were observed. These results strongly suggest that the virus-specific T cell exhaustion seen during persistent HBV infection in patients is not due to repeated systemic activation mediated by circulating APC. Rather, T cell exhaustion is likely the result of persistent presentation of viral antigen in the liver by infected hepatocytes and non-classical APCs, similar to what has been demonstrated in mice. However, the impact that inflammatory conditions have on circulating CD14 MN could not be ruled out. Circulating monocytes are precursors for inflammatory DC. It has been demonstrated that acute viral infections like influenza cause rapid differentiation of blood monocytes to mature DC. This could lead to presentation of the antigen depot and affect the HBV-specific immune response or pathogenesis of chronic HBV infection. Whether this actually occurs and whether this would be advantageous or deleterious remains to be determined.

While there was no ex vivo cross-presentation, the present disclosure clearly show that the antigen stored in CD14 MN of chronic HBV patients could be efficiently presented to virus-specific CD8 T cells upon DC differentiation. Even more important, presentation of circulating antigen by moDC that were administered with an immunomodulator could trigger the expansion of autologous, HBV-specific T cells present in chronic HBV patients. MN have been shown to capture systemic antigen in mice and activate CD4 T cells but this is the first demonstration that persistent antigen present in chronically infected patients can be utilized to stimulate their own virus-specific T cells. In one example, the caveat to exploiting the method of the present disclosure in chronic HBV patients is the level of exhaustion present in the virus-specific T cell compartment. After years of exposure to high levels of viral antigen, T cells either display an exhausted phenotype or are deleted. However, recent data has shown that younger patients (<30 years) still possess HBV-specific T cells and long-term antiviral therapy can, at least partially, restore HBV-specific T cells in adults. Therefore, applying this approach in patients under successful antiviral therapy would likely be the best route to expand endogenous T cells using circulating antigen captured by CD14 MN.

The magnitude of HBV-specific T cell expansion in the autologous moDC experiments was variable among the different patients and lower relative to peptide-expanded cells. This suggests that the capacity of moDC to stimulate autologous HBV-specific T cells can be further augmented. However, blocking PD-L1 & CTLA-4 or using drugs inhibiting Bim-mediated T cell apoptosis failed to boost T cell expansion. In contrast, the method of the present disclosure is able to expand HBV-specific T cells population. 15-DC induced responses in a higher frequency of patients than 4-DC. Differentiation in the presence of immunomodulator IL-15 confers a moDC phenotype that resembles Langerhan cells, which are known to efficiently cross-present antigen and activate CD8 T cells. Thus, there may be a level of flexibility in how monocytes can be targeted to efficiently harness their antigenic depot.

TABLE 1
Patient characteristics for population analysis

| | | Gender | Age | Ethnicity | HBV DNA^{a} | ALT | |
|---|---|---|---|---|---|---|---|
| | Number | (M/F) | (mean) | (Asian/Caucasian) | (IU/ml) | (U/L) | HBeAg+ |
| Healthy | 12 | 10/2 | 40 | 9/3 | NT | NT | NT |
| HBV Low | 14 | 9/5 | 45 | 11/3 | 3.17E+04 | 54 | 6/14 |
| HBV High | 14 | 9/5 | 37 | 11/3 | 1.88E+08 | 115 | 14/14 |

^{a}Data expressed as Median
NT = not tested

TABLE 2
Patient characteristics for MLR experiments

| | | Gender | Age | Ethnicity | HBV DNA^{a} | ALT | |
|---|---|---|---|---|---|---|---|
| | Number | (M/F) | (mean) | (Asian/Caucasian) | (IU/ml) | (U/L) | HBeAg+ |
| Healthy | 5 | 3/2 | 39 | 4/1 | NT | NT | NT |
| HBV Low | 5 | 3/2 | 50 | 5/0 | 4.24E+03 | 43 | 0/5 |
| HBV High | 5 | 3/2 | 43 | 5/0 | 2.54E+08 | 63 | 5/5 |
| Flare | 5 | 5/0 | 43 | 5/0 | 1.40E+06 | 620 | 4/1 |

^{a}Data expressed as Median
NT = not tested

TABLE 3
Patient characteristics for CD14 MN HBsAg staining

| | | Gender | Age | Ethnicity | HBV DNA^{a} | ALT | |
|---|---|---|---|---|---|---|---|
| | Number | (M/F) | (mean) | (Asian/Caucasian) | (IU/ml) | (U/L) | HBeAg+ |
| Healthy | 11 | 9/2 | 40 | 8/3 | NT | NT | NT |
| HBsAg+ | 12 | 11/1 | 43 | 10/2 | 3.00E+04 | 114 | 2/12 |
| HBsAg− | 7 | 6/1 | 45 | 5/2 | 9.31E+05 | 59 | 4/7 |

^{a}Data expressed as Median
NT = not tested

TABLE 4
Patient characteristics for ex vivo cross-presentation

| | | Gender | Age | Ethnicity | HBV DNA^{a} | ALT | |
|---|---|---|---|---|---|---|---|
| | Number | (M/F) | (mean) | (Asian/Caucasian) | (IU/ml) | (U/L) | HBeAg+ |
| Healthy | 4 | 4/0 | 44 | 2/2 | NT | NT | NT |
| HBV | 7 | 4/3 | 41 | 1/6 | 1.45E+06 | 79 | 4/7 |

^{a}Data expressed as Median
NT = not tested

TABLE 5
Patient characteristics for moDC cross-presentation experiments

| | | Gender | Age | Ethnicity | HBV DNA^{a} | ALT | |
|---|---|---|---|---|---|---|---|
| | Number | (M/F) | (mean) | (Asian/Caucasian) | (IU/ml) | (U/L) | HBeAg+ |
| Healthy | 4 | 4/0 | 43 | 2/2 | NT | NT | NT |
| HLA mismatch | 5 | 4/1 | 35 | 5/0 | 1.63E+03 | 65 | 1/5 |
| HLA match | 9 | 8/1 | 42 | 4/5 | 9.63E+03 | 73 | 2/9 |

^{a}Data expressed as Median
NT = not tested

TABLE 6
Patient characteristics for autolgous expansion

| | | Gender | Age | Ethnicity | HBV DNAa | ALT | |
|---|---|---|---|---|---|---|---|
| | Number | (M/F) | (mean) | (Asian/Caucasian) | (IU/ml) | (U/L) | HBeAg+ |
| Peptide Responder | 16 | 13/3 | 46.3 | 10/6 | 1158 | 45 | 5/16 |
| Peptide non-responder | 4 | 2/2 | 51.7 | 1/3 | 200 | 23 | 1/4 |
| 4-DC responder | 6 | 4/2 | 43.3 | 5/1 | 94.5 | 35.5 | 2/6 |
| 4-DC non-responder | 14 | 13/3 | 49.2 | 6/8 | 12965 | 43.2 | 4/14 |
| 15-DC responder | 7 | 6/1 | 48 | 5/2 | 8700 | 44.8 | 4/7 |
| 15-DC nonresponder | 7 | 6/1 | 46.7 | 3/4 | 1158 | 52.1 | 2/7 |

^{a}Data expressed as Median

1. A method of eliciting an immune response to an antigen present endogenously in a mammal, said method comprising administering to said mammal a composition comprising at least one immunomodulator in an amount sufficient for inducing cell differentiation and/or antigen-presenting function of an antigen-presenting cell precursor that has taken up said antigen.

2. The method according to claim 1, wherein said at least one immunomodulator is selected from the group consisting of a colony stimulating factor, a cytokine, a nucleotide, a tumor necrosis factor, a transforming growth factor, an antibody, a recombinant receptor ligand, a chemokine, a carbohydrate, a lipid, a pathogen associated molecular pattern (PAMP); an endogenous danger-associated molecular pattern (DAMP), a CD40 ligand, an ALUM (AB(SO₄)₂12H₂O) (A is an element selected from Na and K; B is an element selected from Al and Cr) and a combination thereof.

3. The method according to claim 2, wherein said colony stimulating factor is selected from the group consisting of granulocyte-macrophage colony-stimulating factor (GM-CSF), granulocyte colony-stimulating factor (G-CSF), a macrophage colony-stimulating factor (M-CSF or CSF-1), and a combination thereof.

4. The method according to claim 2, wherein said cytokine is selected from the group consisting of interleukin-1 (IL-1), IL-2, IL-3, IL-4, IL-6, IL-10, IL-11, IL-12, IL-15, IL-18, IL-32, interferon-α (IFN-α), IFN-γ, and a combination thereof.

5. The method according to claim 2, wherein said nucleotide is selected from the group consisting of a nucleotide comprising a CpG motif that is recognized by TLR-9, a single stranded RNA, imidazoquinolines and analogs thereof, nucleosides and analogs thereof, and a combination thereof.

6. The method according to claim 1, wherein said composition comprises at least two immunomodulators selected from the group consisting of:
(a) GM-CSF and IL-4;
(b) GM-CSF and IFN-α;
(c) GM-CSF and CpG;
(d) GM-CSF and IL-15;
(e) GM-CSF and IFN-γ;
(f) GM-CSF and IL-32;
(g) GM-CSF and LILRA2;
(h) GM-CSF and Alum; and
(i) GM-CSF and CD40L.

7. The method according to claim 1, wherein said amount sufficient for inducing cell differentiation and/or antigen-presenting function of said antigen presenting cell precursor that has taken up said antigen is about 1 ng/mL to about 100 μg/mL.

8. The method according to claim 1, wherein said amount sufficient for inducing cell differentiation and/or antigen-presenting function of said antigen-presenting cell precursor that has taken up said antigen is an amount sufficient to reach a concentration equivalent to between about 1 ng/ml to about 100 ng/ml in vivo.

9. The method according to claim 1, wherein said antigen is selected from the group consisting of an antigen circulating in the peripheral system of said mammal, an antigen present in a tissue of said mammal, and a combination thereof.

10. The method according to claim 1, wherein said antigen is selected from the group consisting of a virus, a parasite, helminths, a fungi, a microorganism, an allergen, a tumour cell, and components thereof.

11. The method according to claim 10, wherein said virus is selected from the group consisting of Hepatitis B virus (HBV), Hepatitis C virus (HCV), Human Immunodeficiency Virus (HIV), Epstein-Bar virus (EBV), human Cytomegalovirus. Herpes Simplex virus (HSV), Measles virus, Rabies virus, and components thereof.

12. The method according to claim 1, wherein said antigen is selected from the group consisting of Hepatitis B surface antigen (HBsAg), Hepatitis B core-antigen (HBcAg), and Hepatitis B e-antigen (HBeAg) and Hepatitis B polymerase antigen (HBpAg).

13. The method according to of claim 1, wherein said antigen-presenting cell precursor is selected from the group consisting of antigen-presenting cell precursors circulating in the peripheral system of said mammal, antigen-presenting cell precursors present in tissues of said mammal, and combinations thereof.

14. The method according to claim 1, wherein said antigen-presenting cell precursor is selected from the group consisting of a monocyte, a macrophage, and tissue resident lineages thereof.

15. The method according to claim 14, wherein said monocyte is CD14 monocyte.

16. The method according to claim 13, wherein said antigen-presenting cell precursor present in tissues of said mammal is selected from the group consisting of liver Kupffer cells and endothelial cells.

17. The method according to claim 1, wherein said antigen-presenting cell precursor is not an antigen-presenting cell precursor selected from the group consisting of B cell myeloid dendritic cell, CD141 dendritic cell, and CD123 plasmacytoid dendritic cell.

18. The method according to claim 1, further comprising administering an activator compound selected from the group consisting of Toll-like Receptor-1 (TLR-1) agonist. TLR- 2 agonist, TLR-3 agonist, TLR-4 agonist, TLR-5 agonist, TLR-6 agonist, TLR-7 agonist. TLR-8 agonist, TLR-9 agonist, TLR-10 agonist, CD-40L agonist, interferon-α (IFN-α) agonist, IFN-β agonist, IFN-γ agonist, PAMPS agonist, DAMPS agonist, an ALUM (AB(SO₄)₂-12H₂O) (A is an element selected from Na and K; B is an element selected from Al and Cr), and a combination thereof.

19. The method according to claim 1, wherein said method induces differentiation of said antigen-presenting cell precursor into dendritic cell.

20. The method according to claim 1, wherein said immune response comprises activation of T cell function.

21. The method according to claim 20, wherein said T cell is selected from the group consisting of CD8 T cell, CD4 T cell, and a combination thereof.

22. The method according to claim 1, wherein said method is for eliciting an immune response to an antigen present endogenously in a mammal suffering from a chronic disease.

23. The method according to claim 22, wherein said chronic disease is selected from the group consisting of Hepatitis B virus (HBV) infection, Hepatitis C virus (HCV) infection, Human Immunodeficiency Virus (HIV) infection, Epstein-Bar virus (EBV) infection, human Cytomegalovirus infection, Herpes Simplex virus (HSV) infection, Measles virus infection, Rabies virus infection, malaria infection, and Helminth infection.

24. The method according to claim 1, wherein the composition does not contain exogenous antigen.

25. A composition for eliciting an immune response to an antigen present endogenously in a mammal, comprising at least one immunomodulator in an amount sufficient for inducing cell differentiation and/or antigen-presenting function of an antigen-presenting cell precursor that has taken up said antigen.

26. The composition according to claim 25, wherein said at least one immunomodulator is selected from the group consisting of a colony stimulating factor, a cytokine, a nucleotide, a tumor necrosis factor, a transforming growth factor, an antibody; a recombinant receptor ligand, a chemokine, a carbohydrate, a lipid, a pathogen associated molecular pattern (PAMP); an endogenous danger-associated molecular pattern (DAMP), a CD40 ligand, an ALUM (AB(SO₄)₂-12H₂O) (A is an element selected from Na and K; B is an element selected from Al and Cr), and a combination thereof.

27. The composition according to claim 26, wherein said colony stimulating factor is selected from the group consisting of granulocyte-macrophage colony-stimulating factor (GM-CSF), granulocyte colony-stimulating factor (G-CSF), a macrophage colony-stimulating factor (M-CSF or CSF-1), and a combination thereof.

28. The composition according to claim 26, wherein said cytokine is selected from the group consisting of interleukin-1 (IL-1), IL-2, IL-3, IL-4, IL-6, IL-10, IL-11, IL-12, IL-15, IL-18, IL-32, interferon-α (IFN-α), IFN-β, IFN-γ, and a combination thereof.

29. The composition according to claim 26, wherein said nucleotide is selected from the group consisting of a nucleotide comprising a CpG motif that is recognized by TLR-9, a single stranded RNA, imidazoquinolines and analogs thereof, nucleosides and analogs thereof, and a combination thereof.

30. The composition according to claim 25, wherein said composition comprises at least two immunomodulators selected from the group consisting of:
(a) GM-CSF and IL-4;
(b) GM-CSF and IFN- [symbol] ;
(c) GM-CSF and CpG;
(d) GM-CSF and IL-15;
(e) GM-CSF and IFN-γ;
(f) GM-CSF and IL-32;
(g) GM-CSF and LILRA2;
(h) GM-CSF and Alum; and
(i) GM-CSF and CD40L.

31. The composition according to claim 25, wherein said amount sufficient for inducing cell differentiation and/or antigen-presenting function of said antigen-presenting cell precursor that has taken up said antigen is about 1 ng/mL to about 100 μg/mL.

32. The composition according to claim 25, wherein said amount sufficient for inducing cell differentiation and/or antigen-presenting function of said antigen-presenting cell precursor that has taken up said antigen is an amount sufficient to reach a concentration equivalent to between about 1 ng/ml to about 100 ng/ml in vivo.

33. The composition according to claim 25, wherein said antigen is selected from the group consisting of an antigen circulating in the peripheral system of said mammal, an antigen present in a tissue of said mammal, and a combination thereof.

34. The composition according to claim 25, wherein said antigen is selected from the group consisting of a virus, a parasite, helminths, a fungi a microorganism, an allergen, a tumor cell, and components thereof.

35. The composition according to claim 34, wherein said virus is selected from the group consisting of Hepatitis B virus (HBV), Hepatitis C virus (HCV), Human Immunodeficiency Virus (HIV), Epstein-Bar virus (EBV), human Cytomegalovirus. Herpes Simplex virus (HSV), Measles virus, Rabies virus, and components thereof.

36. The composition according to claim 25, wherein said antigen is selected from the group consisting of Hepatitis B surface antigen (HBsAg), Hepatitis B core antigen (HBcAg), and Hepatitis B e-antigen (HBeAg) and Hepatitis B polymerase antigen (HBpAg)

37. The composition according to claim 25, wherein said antigen-presenting cell precursor is selected from the group consisting of antigen-presenting cell precursors circulating in the peripheral system of said mammal, antigen-presenting cell precursors present in tissues of said mammal, and combinations thereof.

38. The composition according to claim 25, wherein said antigen-presenting cell precursor is selected from the group consisting of a monocyte, a macrophage, and tissue resident lineages thereof.

39. The composition according to claim 38, wherein said monocyte is CD14 monocyte.

40. The composition according to claim 37, wherein said antigen-presenting cell precursors present in tissues of said mammal are selected from the group consisting of liver Kupffer cells and endothelial cells.

41. The composition according to claim 25, wherein said antigen-presenting cell precursor is not an antigen-presenting cell precursor selected from the group consisting of B cell myeloid dendritic cell, CD141 dendritic cell, and CD123 plasmacytoid dendritic cell.

42. The composition according to claim 25, further comprising an activator compound selected from the group consisting of Toll-like Receptor-1 (TLR-1) agonist, TLR- 2 agonist, TLR-3 agonist, TLR-4 agonist, TLR-5 agonist, TLR-6 agonist, TLR-7 agonist, TLR-8 agonist, TLR-9 agonist, TLR-10 agonist, CD-40L agonist, interferon-α (IFN-α) agonist, IFN-β agonist, IFN-γ agonist, PAM PS agonist, DAMPS agonist, Alum (AB(SO₄)₂-12H₂O) (A is an element selected from Na and K; B is an element selected from Al and Cr); and a combination thereof.

43. The composition according to claim 25, wherein said composition induces differentiation of said antigen-presenting cell precursor into dendritic cell.

44. The composition according to claim 25, wherein said immune response comprises activation of T cell function.

45. The composition according to claim 44, wherein said T cell is selected from the group consisting of CD8 T cell, CD4 T cell, and a combination thereof.

46. The composition according to claim 25, wherein said composition is for eliciting an immune response to an antigen present endogenously in a mammal suffering from a chronic disease.

47. The composition according to claim 46, wherein said chronic disease is selected from the group consisting of Hepatitis B virus (HBV) infection, Hepatitis C virus (HCV) infection, Human Immunodeficiency Virus (HIV) infection, Epstein-Bar virus (EBV) infection, human Cytomegalovirus infection, Herpes Simplex virus (HSV) infection, Measles virus infection, Rabies virus infection, malaria infection, Helminth infection, and fungal infection.

48. The composition according to claim 25, wherein the composition does not contain exogenous antigen.

49. An adjuvant comprising a composition for eliciting an immune response to an antigen present endogenously in a mammal comprising at least one immunomodulator in an amount sufficient for inducing cell differentiation and/or antigen-presenting function of an antigen-presenting cell precursor that has taken up said antigen.

50. An immunomodulator that is capable of eliciting an immune response to an antigen present endogenously in a mammal, wherein said immunomodulator is present in an amount sufficient for inducing cell differentiation and/or antigen-presenting function of an antigen-presenting cell precursor that has taken up said antigen.


SEQUENCE LISTING (1 sequence)

SEQ ID NO: 1 - DNA, 24 bp, artificial sequence
  CpG motif
tcgtcgttttgtcgttttgtcgtt